(19) United States
(12) Patent Application Publication
(10) Pub. No.: US 2026/0139036 A1
(43) Pub. Date: May 21, 2026

(54) CHIMERIC ANTIBODIES AND METHODS OF MAKING AND USING SAME

(71) Applicant: ABBRATECH INC., Branford, CT (US)

(72) Inventor: Michael P. Weiner, Guilford, CT (US)

(21) Appl. No.: 19/121,368

(22) PCT Filed: Oct. 26, 2023
(86) PCT No.: PCT/US2023/077822

§ 371 Date: Apr. 15, 2025

Related U.S. Application Data
(60) Provisional application No. 63/419,573, filed on Oct. 26, 2022.
(60) Provisional application No. 63/578,178, filed on Aug. 23, 2023.

(51) Int. Cl. C07K 16/114; G01N 33/68

(52) U.S. Cl. CPC C07K 16/1145; G01N 33/6854; C07K 2317/24; C07K 2317/31; C07K 2317/35

(57) ABSTRACT

The present disclosure provides antigen binding regions and chimeric antibodies, as well as methods of making and using the same.

### CROSS-REFERENCE TO RELATED APPLICATIONS

The present disclosure claims priority to, and the benefit of, U.S. Provisional Patent Application No. 63/419,573, filed 26 Oct. 2022, and U.S. Provisional Patent Application No. 63/578,178, filed 23 Aug. 2023, each of which is incorporated by reference herein in its entirety for all purposes.

### INCORPORATION BY REFERENCE

In compliance with 37 C.F.R. § 1.52(e)(5), the sequence information contained in electronic file name: ATZ0007PCT_Sequence_Listing_ST26.xml; size 27.1 KB; created 4 Oct. 2023 using WIPO Sequence 2.3.0, is hereby incorporated by reference herein in its entirety.

### FIELD OF THE INVENTION

The present disclosure relates generally to chimeric antibodies, and methods of making and using such antibodies.

### BACKGROUND OF THE INVENTION

Most natural antibodies are bi- or multivalent molecules comprising identical antigen binding sites i.e., monospecific. Bispecific antibodies with defined specificities, however, are artificial molecules and are not found in nature. They must, therefore, be generated by biochemical, molecular, or genetic means.

During the past two decades we have seen a phenomenal evolution of bispecific antibodies for therapeutic applications. Bispecific antibodies have over 100 different formats, including small molecules composed solely of the antigen-binding sites of two antibodies, molecules with an IgG structure, and large complex molecules composed of different antigen-binding moieties often combined with dimerization modules. These may vary in size, arrangement, valency, flexibility and geometry of their binding modules, as well as in their distribution and pharmacokinetic properties.

Two-in-One antibody technology, unlike many other bispecific formats, provides an option for dual targeting antibody therapeutics that can be manufactured in regular IgG or Fab formats. Each dual specific Fab therefore interacts with one of its two antigens at a time. A need exists for the development of a chimeric format in where each heavy and light chain can interact independently with the same epitope. The present disclosure solves this need.

### SUMMARY OF THE INVENTION

The present disclosure provides chimeric antigen binding regions, chimeric antibodies, and antigen binding fragments or portions of chimeric antibodies, as well as methods of making, examining, and using the same. An aspect of the present disclosure relates to an antigen binding region (e.g. chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

In any aspect or embodiment described herein, the first epitope and the second epitope are different epitopes on the same antigen.

In any aspect or embodiment described herein, the first epitope and the second epitope are on different antigens.

In any aspect or embodiment described herein, the first epitope and the second epitope are the same epitope.

Another aspect of the present disclosure relates to an antigen binding region (or a method of making an antigen binding region) of the present disclosure produced by a method or process comprising: expressing an antigen binding region (e.g., a chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

A further aspect of the present disclosure relates to a method of making an antigen binding region of the present disclosure, the method comprising: expressing an antigen binding region (e.g., a chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

In any aspect or embodiment described herein, the method or process further comprises: (a) generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both); (b) examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope; (c) examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope; (d) examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope; (e) expressing one or more (e.g., 1, 2, 3, 4, 5, 6, 7, 8, 9, 10, or more) antigen binding regions (e.g., chimeric antigen binding regions) comprising (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; (f) examining or determining the binding capacity, affinity, avidity, or a combination thereof, of one or more antigen binding regions (e.g., chimeric antigen binding regions) comprising (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; or (g) a combination thereof.

An additional aspect of the present disclosure relates to a chimeric antibody (e.g. antibody, bispecific antibody, or multispecific antibody) or fragment thereof comprising one or more (e.g., 1, 2, 3, 4, 5, 6, 7, 8, 9, 10, or more) antigen binding region, wherein each of the one or more antigen binding region is independently selected from the antigen binding region of the present disclosure.

In any aspect or embodiment described herein, the antibody fragment (e.g., an antigen binding fragment of the chimeric antibody) is selected from: (i) an antigen-binding fragment (Fab), (ii) a Fab prime (Fab′), (iii) a bivalent Fab (F(ab′)₂), (iv) a fragment difficult (Fd), (v) a fragment variable or variable fragment (Fv), (vi) a single-chain fragment variable (scFv), (vii) a disulfide-stabilized fragment variable (dsFv), (viii) a (Fv)₂ fragment, (ix) a linear antibody, and (x) a multibody.

In any aspect or embodiment described herein, the chimeric antibody or fragment thereof comprises two or more (e.g., 2, 3, 4, 5, 6, 7, 8, 9, 10 or more) independently selected antigen binding regions.

In any aspect or embodiment described herein, the antibody or fragment thereof is an antibody.

Another aspect of the present disclosure relates to a chimeric antibody or fragment thereof of the present disclosure produced by a method or process comprising: expressing a chimeric antibody or fragment thereof comprising one or more (e.g., 1, 2, 3, 4, 5, 6, 7, 8, 9, 10, or more) independently selected antigen binding regions of the present disclosure.

An additional aspect of the present disclosure relates to a method of making a chimeric antibody or fragment thereof of the present disclosure, the method comprising: expressing a chimeric antibody or fragment thereof comprising one or more (e.g., 1, 2, 3, 4, 5, 6, 7, 8, 9, 10, or more) independently selected antigen binding regions of the present disclosure.

In any aspect or embodiment described herein, the method or process further comprises: (a) generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both); (b) examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope; (c) examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope; (d) examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope; (e) expressing one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; (f) examining or determining the binding capacity, affinity, avidity, or a combination thereof, of one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; or (g) a combination thereof.

A further aspect of the present disclosure relates to a chimeric antibody (e.g., antibody, bispecific antibody, or multispecific antibody) comprising two or more (e.g., 2, 3, 4, 5, 6, 7, 8, 9, 10, or more) antigen binding regions, wherein each antigen binding region is independently selected from the antigen binding region of the present disclosure.

In any aspect or embodiment described herein, each (e.g., 2, 3, 4, 5, 6, 7, 8, 9, 10, or more) antigen binding region is the same antigen binding region.

In any aspect or embodiment described herein, each (e.g., 2, 3, 4, 5, 6, 7, 8, 9, 10, or more) antigen binding region is a different antigen binding region.

In any aspect or embodiment described herein, each antigen binding region bind the same epitope.

In any aspect or embodiment described herein, each antigen binding region bind a different epitope (e.g., different epitopes on the same protein or epitopes on different proteins).

An aspect of the present disclosure relates to a chimeric antibody of the present disclosure produced by a method or process comprising expressing a chimeric antibody comprising one or more (e.g., 1, 2, 3, 4, 5, 6, 7, 8, 9, 10, or more) independently selected antigen binding regions of the present disclosure.

An additional aspect of the present disclosure relates to method of making a chimeric antibody of the present disclosure, the method comprising: expressing a chimeric antibody comprising one or more (e.g., 1, 2, 3, 4, 5, 6, 7, 8, 9, 10, or more) independently selected antigen binding regions of the present disclosure.

In any aspect or embodiment described herein, the method or process further comprises: (a) generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both); (b) examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope; (c) examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope; (d) examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope; (e) expressing one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; (f) examining or determining the binding capacity, affinity, avidity, or a combination thereof, of one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope; or (g) a combination thereof.

In any aspect or embodiment described herein, generating antigen binding region to the first epitope, the second epitope, or both the first epitope and the second epitope, comprises performing site-directed discovery or immunization for the first epitope, the second epitope, or both the first epitope and the second epitope (e.g., wherein the heavy chain independently binds the epitope, the light chain independently binds the epitope, or both).

In any aspect or embodiment described herein, examining or determining the binding capacity, affinity, avidity, or a combination thereof comprises performing screening or examining the specificity and/or affinity of the one or more antigen binding regions (e.g., an antibody or an antigen-binding fragment or portion thereof), the one or more heavy chain variable regions, the one or more light chain variable regions, or a combination thereof, for the first epitope, the second epitope, or both the first epitope and the second epitope.

An aspect of the present disclosure relates to a chimeric antibody (e.g., antibody, bispecific antibody, or multispecific antibody) comprising: (a) a first heavy chain variable region (VH1) and a first light chain variable region (VL1), wherein the VH1 and the VL1 each bind a first epitope independently; and (b) a second heavy chain variable region (VH2) and second light chain variable region (VL2), wherein the VH2 and the VL2 each bind a second epitope independently.

In any aspect or embodiment described herein, the first epitope and the second epitope are the same.

In any aspect or embodiment described herein, the first epitope and the second epitope are different epitopes on the same antigen.

In any aspect or embodiment described herein, the first epitope and the second epitope are different epitopes on different antigens.

A further aspect of the present disclosure relates to a chimeric antibody (e.g., antibody, bispecific antibody, or multispecific antibody) comprising: (a) a first antigen binding region comprising a first heavy chain variable region (VH1) and a first light chain variable region (VL1), wherein the VH1 and the VL1 each independently bind an epitope on a first antigen; and (b) a second antigen binding region comprising a second heavy chain variable region (VH2) and a second light chain variable region (VL2), wherein the VH2 and the VL2 each independently bind an epitope on an antigen.

In any aspect or embodiment described herein, the VH1 and the VL1 bind different epitopes on the first antigen.

In any aspect or embodiment described herein, the VH1 and the VL1 each independently bind the same epitope on the first antigen.

In any aspect or embodiment described herein, the VH2 and the VL2 bind different epitopes on the antigen.

In any aspect or embodiment described herein, the VH2 and the VL2 each independently bind the same epitope on the antigen.

In any aspect or embodiment described herein, the first antigen and the antigen are the same antigen.

In any aspect or embodiment described herein, the VH2 and the VL2 bind epitopes on different antigens.

In any aspect or embodiment described herein, the first antigen and the antigen are different antigens.

In any aspect or embodiment described herein, the first antigen binding region (e.g., the VH1 and the VL1) are part of a first antigen-binding fragment (Fab), the second antigen binding region (e.g., the VH2 and the VL2) are part of a second antigen-binding fragment (Fab), or a combination thereof.

Yet another aspect of the present disclosure relates to a pharmaceutical composition comprising the antigen binding region of the present disclosure, an antibody of the present disclosure, or an antigen binding fragment of the present disclosure.

An additional aspect of the present disclosure relates to a method of treating a disease or disorder in a subject in need thereof comprising administering the antigen binding region of any the present disclosure, the chimeric antibody or fragment thereof of the present disclosure, the chimeric antibody of the present disclosure, or the pharmaceutical composition of the present disclosure.

Unless otherwise defined, all technical and scientific terms used herein have the same meaning as commonly understood by one of ordinary skill in the art to which this disclosure pertains. Although methods and materials similar to or equivalent to those described herein can be used in the practice of the present disclosure, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention of the present disclosure will be apparent from and encompassed by the following detailed description and claims.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating certain embodiments of the disclosure and are not to be construed as limiting the disclosure. Further objects, features and advantages of the disclosure will become apparent from the following detailed description taken in conjunction with the accompanying figures that show illustrative embodiments of the disclosure.

FIG. 1 illustrates the development of the chimeric antibodies of the present disclosure. During B-cell cloning, heavy and light chains that can bind to a target site independently are often identified.

FIG. 2 illustrates an exemplary cloning workflow that generates wells with many B-cells. Typically, only heavy and light chains from the same well are paired.

FIG. 3 illustrates the potential uses of the chimeric antibodies of the present disclosure. Membrane-proximal external region (MPER) region of Human Immunodeficiency Virus envelope glycoprotein (Env); angiotensin converting enzyme-2 (Ace2); Kirsten rat sarcoma virus (KRAS) missense mutants at position 12 (KRAS G12AX); C—C chemokine receptor type 5 (CCR5).

FIGS. 4A, 4B, and 4C. (4A) Filoviridae GP protein. The filovirus surface glycoprotein (GP) is a trimeric structure that drives viral entry into the host cell via a complex mechanism that is, to date, only partially understood. The GP monomer protein is composed of a glycan cap (orange), and GP1 (blue) and the GP2 (green) subdomains that are cleaved by furin prior to viral entry. Targeted sites 1, 2, and 3 surround the furin cleavage site, “F”, are mapped onto the GP1-GP2 interface. Binding of therapeutic antibodies at sites 1, 2, and 3 are known to block furin cleavage and subsequently, viral entry. (4B) TGF-Beta structure dimer. Targeted sites 1, 2, and 3 surround the furin cleavage site, “F” (Shi M, Zhu J, Wang R, Chen X, Mi L, Walz T, Springer T A. Latent TGF-β structure and activation. Nature. 2011 Jun. 15; 474(7351):343-9. PDB: 3RJR). (4C) Pro-myostatin structure dimer. Targeted sites 1, 2, and 3 surround the furin cleavage site, “F” (Cotton T R, Fischer G, Wang X, McCoy J C, Czepnik M, Thompson T B, Hyvönen M. Structure of the human myostatin precursor and determinants of growth factor latency. EMBO J. 2018 Feb. 1; 37(3):367-383. PDB: 5NTU).

FIGS. 5A and 5B. (5A) Enzyme-linked immunosorbent assay (ELISA) of site-directed discovery (Epivolve) in vitro generated IgGs to modified (Left Panel) and unmodified (Middle Panel) peptides and full-length folded protein (Right Panel). The IgG clones in left panel were isolated by phage display using a peptide immunogen in rounds one, two, and three for both discovery and affinity maturation. They evolved in vitro using error prone polymerase chain reaction (EP-PCR). As expected, since they were affinity matured in vitro against the peptides, all IgGs show strong binding to the mod1 peptide and native peptide. One clone, IgG11 exhibits binding to the folded full-length protein (Right Panel). Clones that failed full length folded binding were tested in denatured full-length (FL) protein ELISA. All showed detectable, but weak binding to denatured full length protein. (5B) anti-proprotein convertase subtilisin/kexin type 9 (PCSK9) binding. ELISA of sited-directed discovery (Epivolve) in vitro to derive monoclonal antibodies (mAbs) that have functionality as an enzyme inhibitor and demonstrates binding to full length, active enzyme. Unlike in FIG. 5A, these clones were affinity matured using EP-PCR and screened against folded PCSK9. Example binding of evolved single chain fragment variables (scFvs) to a full-length PCSK9 protein. The leftmost bar is a negative control that uses no PCSK9 in the assay, and two rightmost are a negative control that uses random (irrelevant) scFv clones. The enzyme-inhibitory activity of clones between the controls were isolated by discovery phage display using mod1 peptides corresponding to an active site adjacent loop in rounds one, two, and three, and then affinity matured against full length, folded PCSK9. Six (A05, E10, B02, E05, D03, G10) of the 8 clones demonstrate a degree of inhibition with two that bind PCSK9 but do not inhibit its activity (C09 and A08).

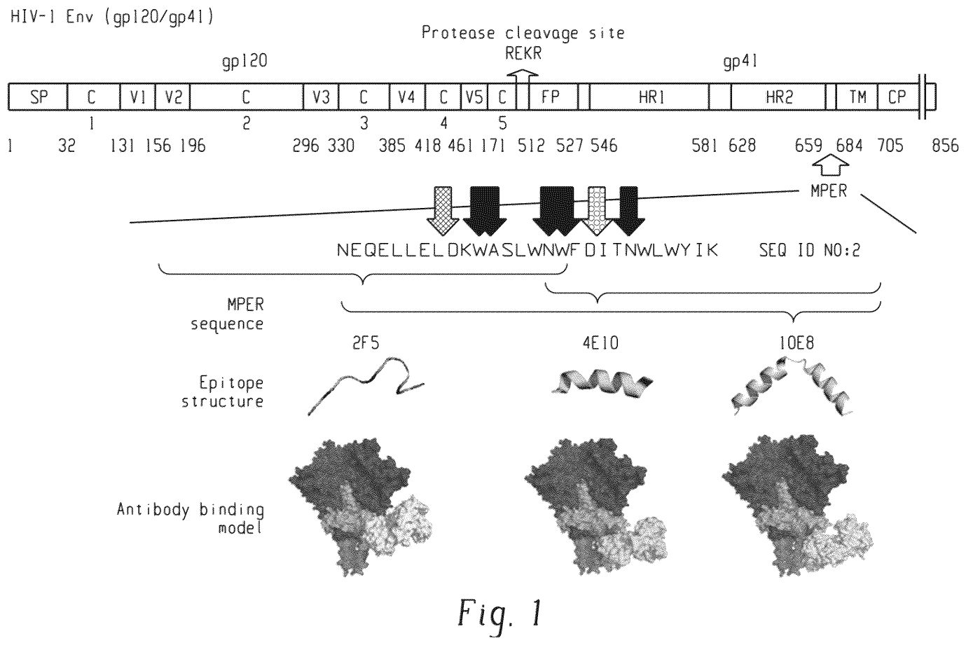

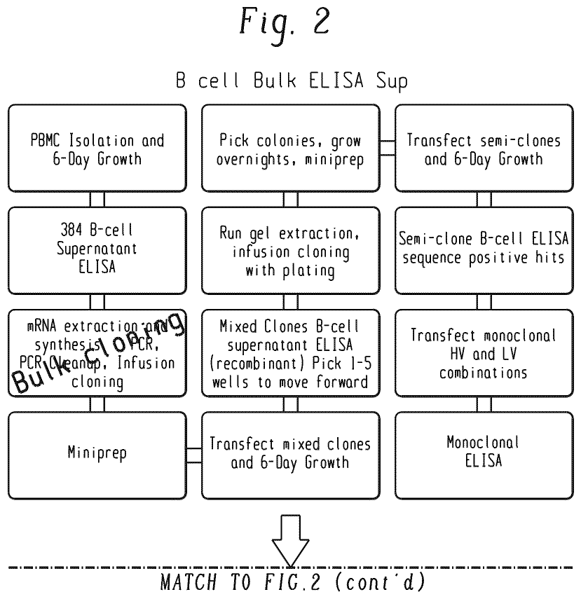

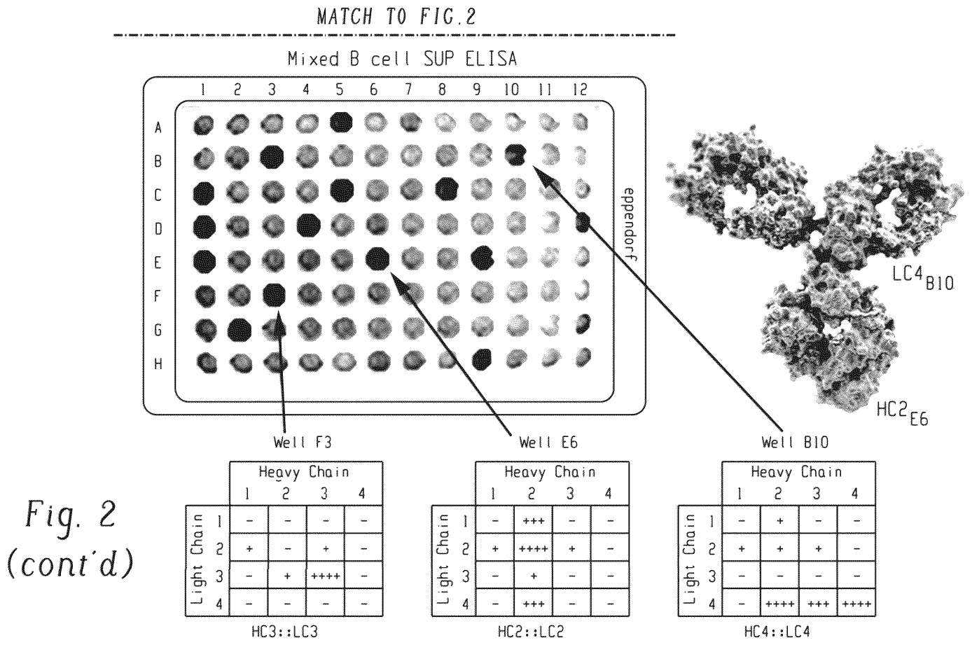

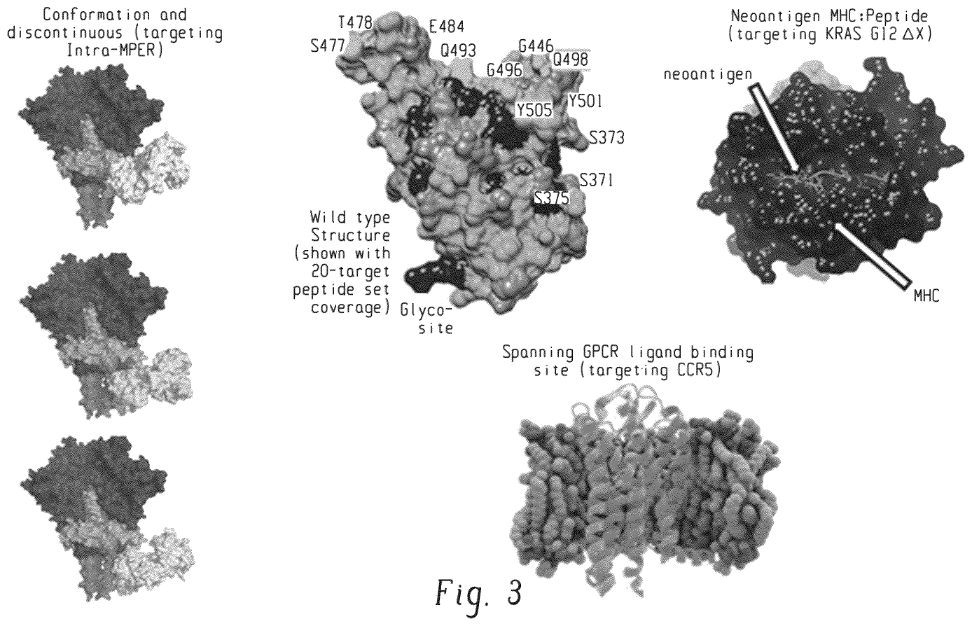

### DETAILED DESCRIPTION OF THE INVENTION

The disclosure provides unique chimeric antibodies where each immunoglobulin chain (heavy and light) pair interacts independently with the same epitope.

The discovery is based upon is based upon the “Epivolve” methodology, which is a unique method of antigen design followed by the either the in vivo exploitation somatic hyper-mutagenesis or in vitro using a method called “discovery maturation” or “DisMat”. If needed, these DisMatted Abs can be affinity matured (AffMatted) for higher affinity to the native peptide or protein.

The disclosure is based in part on the discovery that during B cell cloning of the Epivolve antibodies, heavy and light chains were identified that can bind to the target site (i.e., epitope) independently.

Standard cloning workflow generates wells with many B cells, typically only heavy and light chains from the same well are paired. In contrast, the present disclosure identifies heavy and light chains that can independently bind the target site and pairs independently derived heavy chains with independently derived light chain. Surprisingly, these pairings resulted in antibodies with increased affinity due to an apparent avidity effect.

The antibodies produced by the methods described herein are called Frankenstein antibodies (“FnAbs”). FnAbs can be used for example top develop antibodies to confirmational and discontinuous epitopes; antibodies that can bind across a binding pocket; antibodies against neoantigen; or antibodies that can span a transmembrane binding site (e.g., GPCR ligand binding site).

Accordingly, the present disclosure provides a chimeric antibody having a heavy and light chain that can bind an epitope independently. In some embodiments the disclosure provides an antibody having a first heavy chain variable region (VH1), and a first light chain variable region (VL1) where the VH1 and VL1 each bind a first epitope independently; and a second heavy chain variable region (VH2), and second light chain variable region (VL2), where the VH2 and VL2 each bind a second epitope independently. The first and second epitope are the same epitope. Alternatively, the first and second epitopes are different epitopes on the same protein. In other embodiments the first and second epitopes are different epitopes on the same protein. FnAbs developed using the disclosure have application in a wide variety of fields such as for example, in the development of vaccines, diagnostics, biosimilars, CAR-Ts; therapeutics, bispecific and multispecific antibodies.

### Chimeric Antigen Binding Regions or Chimer Antibodies (or Antigen-Binding Fragments or Portions Thereof) of the Present Disclosure

The present disclosure provides chimeric antigen binding regions, chimeric antibodies, and antigen binding fragments or portions of chimeric antibodies, as well as methods of making, examining, and using the same. An aspect of the present disclosure relates to an antigen binding region (e.g. chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

In any aspect or embodiment described herein, the first epitope and the second epitope are different epitopes on the same antigen. In any aspect or embodiment described herein, the first epitope and the second epitope are on different antigens. In any aspect or embodiment described herein, the first epitope and the second epitope are the same epitope.

An additional aspect of the present disclosure relates to a chimeric antibody (e.g. antibody, bispecific antibody, or multispecific antibody) or fragment thereof comprising one or more (e.g., 1, 2, 3, 4, 5, 6, 7, 8, 9, 10, or more) antigen binding region, wherein each of the one or more antigen binding region is independently selected from the antigen binding region of the present disclosure.

In any aspect or embodiment described herein, the antibody fragment (e.g., an antigen binding fragment of the chimeric antibody) is an antigen-binding fragment (Fab). In any aspect or embodiment described herein, the antibody fragment (e.g., an antigen binding fragment of the chimeric antibody) is a Fab prime (Fab′). In any aspect or embodiment described herein, the antibody fragment (e.g., an antigen binding fragment of the chimeric antibody) is a bivalent Fab (F(ab′)₂). In any aspect or embodiment described herein, the antibody fragment (e.g., an antigen binding fragment of the chimeric antibody) is a fragment difficult (Fd). In any aspect or embodiment described herein, the antibody fragment (e.g., an antigen binding fragment of the chimeric antibody) is a fragment variable or variable fragment (Fv). In any aspect or embodiment described herein, the antibody fragment (e.g., an antigen binding fragment of the chimeric antibody) is a single-chain fragment variable (scFv). In any aspect or embodiment described herein, the antibody fragment (e.g., an antigen binding fragment of the chimeric antibody) is a disulfide-stabilized fragment variable (dsFv). In any aspect or embodiment described herein, the antibody fragment (e.g., an antigen binding fragment of the chimeric antibody) is a (Fv)₂ fragment. In any aspect or embodiment described herein, the antibody fragment (e.g., an antigen binding fragment of the chimeric antibody) is a linear antibody. In any aspect or embodiment described herein, the antibody fragment (e.g., an antigen binding fragment of the chimeric antibody) is a multibody.

In any aspect or embodiment described herein, the chimeric antibody or fragment thereof comprises two or more (e.g., 2, 3, 4, 5, 6, 7, 8, 9, 10 or more) independently selected antigen binding regions. In any aspect or embodiment described herein, the antibody or fragment thereof is an antibody.

A further aspect of the present disclosure relates to a chimeric antibody (e.g., antibody, bispecific antibody, or multispecific antibody) comprising two or more (e.g., 2, 3, 4, 5, 6, 7, 8, 9, 10, or more) antigen binding regions, wherein each antigen binding region is independently selected from the antigen binding region of the present disclosure.

In any aspect or embodiment described herein, each (e.g., 2, 3, 4, 5, 6, 7, 8, 9, 10, or more) antigen binding region is the same antigen binding region. In any aspect or embodiment described herein, each (e.g., 2, 3, 4, 5, 6, 7, 8, 9, 10, or more) antigen binding region is a different antigen binding region. In any aspect or embodiment described herein, each antigen binding region bind the same epitope. In any aspect or embodiment described herein, each antigen binding region bind a different epitope (e.g., different epitopes on the same protein or epitopes on different proteins).

An aspect of the present disclosure relates to a chimeric antibody (e.g., antibody, bispecific antibody, or multispecific antibody) comprising: (a) a first heavy chain variable region (VH1) and a first light chain variable region (VL1), wherein the VH1 and the VL1 each bind a first epitope independently; and (b) a second heavy chain variable region (VH2) and second light chain variable region (VL2), wherein the VH2 and the VL2 each bind a second epitope independently.

In any aspect or embodiment described herein, the first epitope and the second epitope are the same. In any aspect or embodiment described herein, the first epitope and the second epitope are different epitopes on the same antigen. In any aspect or embodiment described herein, the first epitope and the second epitope are different epitopes on different antigens.

A further aspect of the present disclosure relates to a chimeric antibody (e.g., antibody, bispecific antibody, or multispecific antibody) comprising: (a) a first antigen binding region comprising a first heavy chain variable region (VH1) and a first light chain variable region (VL1), wherein the VH1 and the VL1 each independently bind an epitope on a first antigen; and (b) a second antigen binding region comprising a second heavy chain variable region (VH2) and a second light chain variable region (VL2), wherein the VH2 and the VL2 each independently bind an epitope on an antigen.

In any aspect or embodiment described herein, the VH1 and the VL1 bind different epitopes on the first antigen. In any aspect or embodiment described herein, the VH1 and the VL1 each independently bind the same epitope on the first antigen.

In any aspect or embodiment described herein, the VH2 and the VL2 bind different epitopes on the antigen. In any aspect or embodiment described herein, the VH2 and the VL2 each independently bind the same epitope on the antigen. In any aspect or embodiment described herein, the VH2 and the VL2 bind epitopes on different antigens.

In any aspect or embodiment described herein, the first antigen and the antigen are the same antigen. In any aspect or embodiment described herein, the first antigen and the antigen are different antigens.

In any aspect or embodiment described herein, the first antigen binding region (e.g., the VH1 and the VL1) are part of a first antigen-binding fragment (Fab), the second antigen binding region (e.g., the VH2 and the VL2) are part of a second antigen-binding fragment (Fab), or a combination thereof.

### Methods of Making Antigen Binding Regions or Antibodies (or Antigen-Binding Fragments or Portions Thereof) of the Present Disclosure

Another aspect of the present disclosure relates to an antigen binding region (or a method of making an antigen binding region) of the present disclosure produced by a method or process comprising: expressing an antigen binding region (e.g., a chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

A further aspect of the present disclosure relates to a method of making an antigen binding region of the present disclosure, the method comprising: expressing an antigen binding region (e.g., a chimeric antigen binding region) comprising a heavy chain variable region (e.g., a first heavy chain variable region (VH1) or a second heavy chain variable region (VH2)) and a light chain variable region (e.g., a first light chain variable region (VL1) or a second light chain variable region (VL2)), wherein the heavy chain variable region independently binds a first epitope and the light chain variable region independently binds a second epitope.

In any aspect or embodiment described herein, the method or process further comprises generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both).

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope.

In any aspect or embodiment described herein, the method or process further comprises expressing one or more (e.g., 1, 2, 3, 4, 5, 6, 7, 8, 9, 10, or more) antigen binding regions (e.g., chimeric antigen binding regions) comprising (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

In any aspect or embodiment described herein, the method or process further comprises examining or determining the binding capacity, affinity, avidity, or a combination thereof, of one or more antigen binding regions (e.g., chimeric antigen binding regions) comprising (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

Another aspect of the present disclosure relates to a chimeric antibody or fragment thereof of the present disclosure produced by a method or process comprising: expressing a chimeric antibody or fragment thereof comprising one or more (e.g., 1, 2, 3, 4, 5, 6, 7, 8, 9, 10, or more) independently selected antigen binding regions of the present disclosure.

An additional aspect of the present disclosure relates to a method of making a chimeric antibody or fragment thereof of the present disclosure, the method comprising: expressing a chimeric antibody or fragment thereof comprising one or more (e.g., 1, 2, 3, 4, 5, 6, 7, 8, 9, 10, or more) independently selected antigen binding regions of the present disclosure.

In any aspect or embodiment described herein, the method or process further comprises generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both).

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope.

In any aspect or embodiment described herein, the method or process further comprises expressing one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

An aspect of the present disclosure relates to a chimeric antibody of the present disclosure produced by a method or process comprising expressing a chimeric antibody comprising one or more (e.g., 1, 2, 3, 4, 5, 6, 7, 8, 9, 10, or more) independently selected antigen binding regions of the present disclosure.

An additional aspect of the present disclosure relates to method of making a chimeric antibody of the present disclosure, the method comprising: expressing a chimeric antibody comprising one or more (e.g., 1, 2, 3, 4, 5, 6, 7, 8, 9, 10, or more) independently selected antigen binding regions of the present disclosure.

In any aspect or embodiment described herein, the method or process further comprises generating (e.g., generating by site-directed discovery/immunization) antigen binding region (e.g., an antibody or an antigen-binding fragment or portion thereof) to the first epitope, the second epitope, or both the first epitope and the second epitope (e.g., wherein the heavy chain independently binds the epitope, the light chain independently binds the epitope, or both).

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) antigen binding region (e.g., an antibody or an antigen-binding fragment or portion thereof) for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) heavy chain variable regions for the first epitope, the second epitope, or both the first epitope and the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) light chain variable regions for the first epitope, the second epitope, or both the first epitope and the second epitope.

In any aspect or embodiment described herein, the method or process further comprises expressing one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

In any aspect or embodiment described herein, the method or process further comprises examining or determining the binding capacity, affinity, avidity, or a combination thereof, of one or more (e.g., 1, 2, 3, 4, 5, 6, 7, 8, 9, 10, or more) antigen binding regions (e.g., chimeric antigen binding regions), antibody (e.g., chimeric homodimer antibody) comprising two antigen binding regions, antibody fragment comprising one or more antigen binding regions, or combination thereof, wherein each antigen binding region comprises (i) a heavy chain variable region (e.g., a first heavy chain variable region (VH1) or a second heavy chain variable region (VH2)) that independently binds the first epitope and (ii) a light chain variable region (e.g., a first light chain variable region (VL1) or a second light chain variable region (VL2)) that independently binds the second epitope.

In any aspect or embodiment described herein, generating antigen binding region to the first epitope, the second epitope, or both the first epitope and the second epitope, comprises performing site-directed discovery or immunization for the first epitope, the second epitope, or both the first epitope and the second epitope (e.g., wherein the heavy chain independently binds the epitope, the light chain independently binds the epitope, or both).

In any aspect or embodiment described herein, screening or examining the specificity and/or affinity of the one or more antigen binding regions (e.g., an antibody or an antigen-binding fragment or portion thereof), the one or more heavy chain variable regions, the one or more light chain variable regions, or a combination thereof, for the first epitope, the second epitope, or both the first epitope and the second epitope.

In any aspect or embodiment described herein, the method or process further comprises screening or examining specificity and/or affinity of the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, for the first epitope, the second epitope, or both the first epitope and the second epitope, comprising: (1) providing a set of fusion proteins that comprises two or more (e.g., 2, 3, 4, 5, 6, 7, 8, 9, 10, 11, 12, 13, 14, 15, 16, 17, 18, 19, or 20) fusion proteins, wherein: (a) each fusion protein includes a maltose-binding protein, or amylose-binding derivative thereof, fuses directly to or via a linker to a target sequence of about 8 amino acids to 18 amino acids (e.g., about 10 to about 18 amino acids, about 10 to about 16 amino acids, or 8, 9, 10, 11, 12, 13, 14, 15, 16, 17, or 18 amino acids) from the first epitope and/or the second epitope; and (b) each target sequence of the set of fusion proteins has a different proteinogenic amino acid at a site of interest in the target sequence, including (i) one that has the native amino acid of the target sequence or (ii) one has a native amino acid when the site of interest is a variant site; (2) examining or detecting which of the fusion proteins that the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, binds.

In any aspect or embodiment described herein, detecting is performed via an enzyme-linked immunosorbent assay, and each fusion protein is a different antigen of the enzyme-linked immunosorbent assay (e.g., each fusion protein is located in a different well).

In any aspect or embodiment described herein, (1) the maltose-binding protein derivative has been engineered/modified to provide tighter binding (e.g., enhance the binding) to amylose resin; (2) the linker is a protein linker; (3) the linker is a protein linker, wherein the protein linker includes or consists of about 1 to about 10 amino acids (e.g., 1, 2, 3, 4, 5, 6, 7, 8, 9, or 10 amino acids, each amino acid is independently selected from glycine and serine (such as a GS, GSG, GSGS (SEQ ID NO:1), GSGSG (SEQ ID NO:2), or GSGSGS (SEQ ID NO:3) linker), or a combination thereof); (4) the target sequence is linked to the C-terminus of the maltose-binding protein; or (5) a combination thereof.

In any aspect or embodiment described herein, the set of fusion proteins includes a fusion protein comprising a target sequence for two or more variants of the first epitope and/or the second epitope (e.g., each variant of the first epitope and/or second epitope).

In any aspect or embodiment described herein, the antigen binding region binds to a specific variant of the protein or peptide of interest (e.g., a variant sited antigen binding region, antibody, or antigen binding fragment or portion thereof).

In any aspect or embodiment described herein, the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, binds to a specific set of (e.g., 1, 2, 3, 4, 5, 6, or more) variants (e.g., all variants) of the first epitope and/or the second epitope (e.g., a pan variant antigen binding region, antibody or antigen binding fragment or portion thereof, heavy chain variable region, and/or light chain hypervariable region).

In any aspect or embodiment described herein, the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, binds to only target sequence (e.g., the first epitope and/or the second epitope) with the native amino acid at the site of interest (e.g., a sited antigen binding region, antibody or antigen binding fragment or portion thereof, heavy chain variable region, and/or light chain variable region).

In any aspect or embodiment described herein, screening or examining specificity and/or affinity of the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, for the first epitope, the second epitope, or both the first epitope and the second epitope, further comprises: expressing each fusion protein via an expression vector or construct (e.g., plasmid) that expresses the fusion protein (e.g., expressing in bacteria (such as Escherichia coli), fungi, or eukaryotic cell (such as mammalian cell)); purifying each fusion protein from a cell (e.g., bacteria (such as Escherichia coli), fungi, or eukaryotic cell (such as mammalian cell)) in which it was expressed; or a combination thereof.

In any aspect or embodiment described herein, purifying comprises, for one or more of the fusion proteins (e.g., each fusion protein): (a) adding a cell lysate containing the fusion protein to a gravity flow column comprising amylose resin (e.g., the cell lysate that flows through the gravity flow column is collected and added to the gravity glow column one or more (e.g., 1, 2, 3, or 4) times); (b) optionally washing the gravity flow column after the cell lysate is added; (c) eluting and/or collecting the fusion protein; or (d) a combination thereof.

In any aspect or embodiment described herein, screening or examining specificity and/or affinity of the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, for the first epitope, the second epitope, or both the first epitope and the second epitope, further comprises determining or quantifying the lower limit of detection of the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, by examining the first epitope, the second epitope, or both the first epitope and the second epitope, at a plurality of concentrations/amount (e.g., the lower limit of detection is the lowest concentration/amount of the first epitope and/or the second epitope that is detected and a lower concentration/amount is not detected).

In any aspect or embodiment described herein, screening or examining specificity and/or affinity of the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof, for the first epitope, the second epitope, or both the first epitope and the second epitope, further comprises generating the one or more antigen binding regions (e.g., a chimeric antigen binding region, an antibody or an antigen-binding fragment or portion thereof, or a chimeric antibody or an antigen-binding fragment or portion thereof), the one or more heavy chain variable regions, the one or more light chain variable regions, or a combination thereof.

Any method known in the art for generating heavy and light chains capable of binding an epitope, such as the same epitope, can be used to produce chimeric antibodies or FnAbs of the present disclosure.

The methods which have been termed “Epivolve” (See, U.S. Ser. No. 17/954,037, published as U.S. Patent Application Publication No. 2023/02112271 A1, U.S. Ser. No. 63/415,105, U.S. Ser. No. 63/513,064, and Fuller E P, et al. Derivation of splice junction-specific antibodies using a unique hapten targeting strategy and directed evolution. N Biotechnol. 2022 Nov. 25; 71:1-10, each of which is incorporated by reference herein in its entirety for all purposes) and “Sundae” (See, U.S. Ser. No. 63/415,105 and U.S. Ser. No. 63/513,064, each which is incorporated by reference herein in its entirety for all purposes) are particularly suited for the generation of chimeric antigen binding regions and chimeric antibodies (FnAbs).

“Epivolve” allows for precisely targeting the immune system against any pre-determined site(s) on a protein target. Specifically, Epivolve allows for the generation of site-specific antibodies (ssAbs) can be exploited to make “site directed antibodies” (sited Abs). Specifically, once ssAbs are generated they can be matured to recognize the naturally occurring amino acid (i.e., the synonymous amino acid) or a variant amino acid (i.e., a non-synonymous amino acid). The terms “Epivolve” and “site-specific immunization method” are used interchangeably throughout the present disclosure.

“Sundae” is a screening methodology that allows for the identification of antibodies that can specifically bind two or more different amino at a defined variant site within the antibody binding site. The terms “Sundae” and “method for examining specificity and/or affinity of an antigen region” (or antibody) are used interchangeably throughout the present disclosure.

Thus, utilizing “Epivolve” and “Sundae”, immunoglobulin heavy and light chains from different B-cell clones can be paired to form the FnAbs described herein. For example, in any aspect or embodiment described herein, the method or process of making FnAbs includes the site-specific immunization method described herein. By way of further example, in any aspect or embodiment described herein, the method or process of making FnAbs includes the method for examining specificity and/or affinity of an antigen region.

As used therein, the terms “protein of interest”, “polypeptide of interest”, “target protein”, “target polypeptide”, and the like are used interchangeably and include, e.g., antigen, first antigen, second antigen, epitope, first epitope, second epitope, and the like.

The site-specific immunization method (Epivolve) is based upon a unique antigen design. The antigens are designed such that they can be used in vivo as an immunogen to first induce a sited specific antibody immune response or utilized in an in vitro screening/biopanning. In any aspect or embodiment described herein, the site-specific immunization method (Epivolve) employs the use of three peptides: a first peptide (the “nnAA peptide”), a second peptide (NATive 2), and a third peptide NATive 3).

The site-specific immunization method (Epivolve) starts with the replacement of a native amino acid in an initial peptide or protein immunogen with a specifically modified non-native or non-naturally occurring amino acid (nnAA). The modified peptide or protein is used in vivo as an immunogen or in vitro as the substrate-bound target for phage display biopanning.

In vivo, the site-specific immunization method (Epivolve) can be used to exploit somatic hyper-mutagenesis to produce an antibody that specifically recognize the natural occurring variant sites.

In vitro, clones of antibodies biased against the nnAA site and its adjacent context sequence are isolated. In phage display, first the nnAA peptide is screened/biopanned against a naïve variant antibody “discovery” library to identify nnAA-peptide specific antibodies or antigen binding fragment thereof, such as scFvs. These antibodies or antigen binding fragment thereof, such as scFv, parentals are then epivolved using a method called “discovery maturation” or “DisMat”. DisMat uses AXM mutagenesis under our initial discovery conditions against the native variant peptide or native variant protein. If needed, these DisMatted antibodies or antigen binding fragment thereof can be affinity matured (AffMatted) for higher affinity to the native peptide or protein.

nnAA Peptide Design

The nnAA peptide utilizes a non-native or non-naturally occurring amino acid (nnAA) to generate a site-specific immune response. In any aspect or embodiment described herein, the non-naturally occurring amino acid include or is, for example, phosphoserine (SEP), phosphothreonine, or phosphotyrosine.

The non-naturally occurring serves as a “pseudohapten” and from a nnAA-specific antibody can be produced with the foreknowledge that the location of the antibody binding site overlaps or is adjacent to the non-naturally occurring amino acid (nnAA) and directly affected by the non-naturally occurring amino acid (nnAA).

The position of the nnAA is dependent upon the specific application of the sited antibody. For example, in any aspect or embodiment described herein, for splice-site specific antibodies the non-naturally occurring amino acid (nnAA) is offset by +/−2 position relative to the splice site (position 0), such that the antibodies can react with amino acids on both sides of the neojunction. Similarly, in any aspect or embodiment described herein, for post-translationally modified site-specific antibodies, the non-naturally occurring amino acid (nnAA) is offset by +/−1, +/−2 or +/−3 relative to the post-translational modification side (position 0), such as a glycosylation site.

Preferably, in any aspect or embodiment described herein, the non-naturally occurring amino acid (nnAA) is phosphoserine (SEP). Phosphoserine is inexpensive to incorporate into synthetic peptides, particularly systems to incorporate SEP into proteins expressed in Escherichia coli. However, other non-naturally occurring amino acids (nnAA) may be used, such as other charged modifications including, but not limited to, acetylate, isocyanate, sulfate, or nitrate.

In any aspect or embodiment described herein, the nnAA peptide is approximately 10 to 20 amino acids in length. For example, in any aspect or embodiment described herein, the nnAA peptide is 10, 11, 12, 13, 14, 15, 16, 17, 18, 19, 20, or more amino acids in length.

The amino acid sequence of the nnAA peptide is determined by identifying the amino acid or posttranslational modification location (i.e., “the target site”) in the protein of interest (e.g., the antigen, the first antigen, the second antigen, etc.) to which it is desired that the sited antibody is to be directed toward. The target site is referred to herein as position 0. Once the target site is identified, the nnAA peptide is produced by replacing the amino acid residue at the target site or at a position offset with respect to the target site with a non-naturally occurring amino acid (nnAA). In any aspect or embodiment described herein, the offset can be −4, −3, −2, −1, +1, +2, +3, or +4 relative to the target site “0”. The non-naturally occurring amino acid (nnAA) is flanked on both sides with context amino acids (i.e., the “context sequence”) that are identical to the amino acid sequence on both sides of the target site or offset position. Depending upon the length of the nnAA peptide, the amino acid terminus and the carboxy terminus context sequence are both, and independently, about 5, 6, 7, 8, 9, or 10 amino acids in length.

In any aspect or embodiment described herein, to target a conformational epitope, two nnAA peptides (nnAA1 and nnAA2) are produced/utilized, each targeting a different portion of the desired epitope, wherein nnAA1 and nnAA2 are joined or covalently linked together by a linker to form an nnAA1—nnAA1 peptide.

In any aspect or aspect described herein, to target a variant, two nnAA peptides (nnAA1 and nnAA2) are produced/utilized, each targeting a different amino acid.

In any aspect or embodiment described herein, one nnAA peptide (nnAA1) and one native or naturally occurring (nAA) peptide (nAA2) is joined or covalently linked by a linker to form a nnAA1-nAA2 peptide.

In any aspect or embodiment described herein, one nnAA (nnAA2) peptide and one native or naturally occurring (nAA) peptide (nAA1) is joined or covalently linked by a linker to form a nnAA2-nAA1 peptide.

### NATive 2 and NATive 3 Peptide Design

NATive 2 is identical to the nnAA peptide except that the modified amino acid is replaced by the native or naturally occurring amino acid (nAA) (i.e., a nAA peptide) and has 2, 3, 4, 5, or more additional amino acid residues at the amino terminus and carboxyl terminus of the context sequence. These additional amino acids do not correspond to the corresponding amino acids of the protein of interest (e.g., antigen, the first antigen, and/or the second antigen).

NATive 3 is identical to NATive 2 with the exception that the 2, 3, 4, 5, or more additional amino acid residues at the amino terminus and carboxyl terminus of the context sequence are different from NATive 2. These additional amino acids do not correspond to the corresponding amino acids of the protein of interest (e.g., antigen, the first antigen, and/or the second antigen).

In any aspect or embodiment described herein, to target confirmation epitopes, the nAA1 peptide and the nAA2 peptide are joined or covalently linked together by a linker that is different from the linker used in the nnAA peptides to form NATive-NATive 2 peptide. The amino terminus and the carboxyl terminus of NATive-NATive 2 has 2, 3, 4, 5, or more additional amino acid residues that are different from each other and do not correspond to the corresponding amino acid of the protein of interest (e.g., antigen, the first antigen, and/or the second antigen). In any aspect or embodiment described herein, the Native-Native 2 linker is different from the nnAA peptides and/or NATive-NATive 3.

In any aspect or embodiment described herein, Native-Native 3 peptide is formed by joining nAA1 and nAA2 with a different linker from NATive-NATive 2 and the nnAA peptides. The amino terminus and the carboxyl terminus of the NATive-NATive 3 peptide has 2, 3, 4, 5, or more additional amino acid residues that are different from each other, do not correspond to the corresponding amino acid of the protein of interest (e.g. antigen, the first antigen, and/or the second antigen), and are different from that of NATive-NATive 2. In any aspect or embodiment described herein, the Native-Native 3 linker is different from the nnAA peptides and/or NATive-NATive 2.

In any aspect or embodiment described herein, the linker is a peptide linker. For example, in any aspect or embodiment described herein, the amino acid sequences at the amino and carboxyl terminus of peptides (such as NATive peptides) can be, for example, SerGlySer, GlySerGly, GlyGlyGly, or SerSerSer.

In any aspect or embodiment described herein, peptides containing the non-naturally occurring amino acid (nnAA) are produced in an organism (e.g., bacteria, yeast, or mammalian system) capable of incorporating the non-naturally occurring amino acids. Organisms capable of incorporating the non-naturally occurring amino acids are known in the art. For example, an E. coli stain engineered to incorporate a phosphoserine at an amber stop codon or an E. coli strain that incorporates a modified tyrosine that can be changed to a phosphotyrosine in vitro, or a bacterium that is an O-phosphoserine-incorporating suppressing bacterium may be utilized. This immunogen could be a peptide fragment, a peptide genetically fused to a protein (e.g., a highly expressed protein), or the full-length protein itself.

Site specific or sited (sited) antibodies or variant sited (vsited) antibodies can be produced in vivo, by immunizing an animal with the nnAA peptides, or in vitro, by using the nnAA peptides to screen an antibody display library.

In any aspect or embodiment described herein, an animal is immunized by administering the nnAA peptide, NATive 2 peptide, and the protein of interest (e.g., antigen, the first antigen, and/or the second antigen) in its native three-dimensional form (i.e., folded form) (referred to herein as native protein or NP).

In any aspect or embodiment described herein, nnAA peptide, NATive 2 peptide, the NP, or a combination thereof, is administered with or conjugated to one or more (e.g., 1, 2, 3, 4, 5, or more) carriers (e.g., a carrier protein or peptide, such as keyhole limpet hemocyanin (KLH), ovalbumin, or both).

In any aspect or embodiment described herein, immunizing an animal comprises either: (i) administering the nnAA peptide at least once (e.g., one, two, three, or more times), (ii) administering the NATive2 at least once (e.g., one, two, three, or more times), separately or simultaneously, and (iii) optionally, administering the NP at least once (e.g., one, two, three, or more times) after the nnAA peptide and the NATive2 peptide are administered.

In any aspect or embodiment described herein, each immunization/administration (e.g., the nnAA peptide, the NATive 2 peptide, and/or the NP, when administered, is administered at least two weeks apart (e.g., each is administered two weeks to 6 months apart from the prior administration and the subsequent administration).

In any aspect or embodiment described herein, the nnAA peptide, the NATive 2 peptide, the NP, or a combination thereof, is administered with an adjuvant (e.g., Complete Freund's Adjuvant (CFA), Incomplete Freund's Adjuvant (IFA), aluminum, monophosphoryl lipid A (MPL) and aluminum salt (AS04), oil-in-water emulsion, oil-in-water emulsion of squalene (MF59), AS03 (Vitamin E, Surfactant polysorbate 80, and squalene), MPL and QS-21 in a liposome formulation (AS01), cytosine phosphoguanine (CpG), or a combination thereof).

In any aspect or embodiment described herein, the animal is, for example, a primate, (e.g., a human or a non-human primate), a rabbit, a chicken, a rat, a goat, cow, pig, or a mouse. In any aspect or embodiment described herein, the animal is a non-human animal that has a human or humanized immune system.

In any aspect or embodiment described herein, the method further comprises after immunizing (e.g., 5, 6, 7, 8, 9, 10, 11, 12, 13, 14, 15, 16, 17, 18, 19, 20, or more days post final immunization), obtaining/isolating B-cells from the animal. In any aspect or embodiment described herein, the B-cells are obtained from peripheral blood mononuclear cells (PBMCs), splenocytes. bone marrow, or any lymphoid tissue known to contain B-cells. In any aspect or embodiment described herein, the B-cells are cloned (e.g., by any method known in the art).

In any aspect or embodiment described herein, B-cell clones producing site specific or sited antibodies or variant sited antibodies of interest are identified by selecting monoclonal antibodies (mAbs) that do not bind to an appropriate set of negative controls (e.g., a scrambled peptide sequence and/or any carrier protein(s) used in the immunizations) and specifically binds to nnAA peptide, the NATive 2 peptide, and, NATive 3 peptide, as the monoclonal antibodies are presumed to be binding to the common amino acid central core. Additionally, because they bind both (i) nnAA and (ii) the unmodified peptide sequences of the NATive 2 peptide and the NATive 3 peptide the monoclonal antibodies are deemed “nnAA-status independent”—i.e., their binding is independent of the nnAA. In any aspect or embodiment described herein. In any aspect or embodiment described herein, the monoclonal antibodies that bind to both the NATive 2 peptide and the NATive 3 peptide, but not to nnAA peptide are deemed to be binding to the central core but are “native-sequence specific.”

In any aspect or embodiment described herein, site specific or sited antibodies or variant sited antibodies are from a phage display library by sequentially screening the library for binding to the nnAA peptide, the NATive 2 peptide, and the NATive 3 peptide, and generating a library of clonotypes that bind all three, as these antibodies are presumed to be binding to the common amino acid central core.

In any aspect or embodiment described herein, preparing the one or more FnAbs includes: (i) sequencing the heavy and light chain of site specific or sited antibodies or variant sited antibodies identified by the methods herein, (ii) cloning the site specific or sited antibodies or variant sited antibodies heavy chain and light chain in separate expression plasmids; (iii) pairing individual heavy chain and light chain expression plasmids, (iv) expressing the paired expression plasmid, (v) purifying the expressed Fn antibodies, or (vi) a combination thereof.

In any aspect or embodiment described herein, detecting the binding capacity of an antibody produced by the methods described herein (e.g., specific binding) is determined by any appropriate method. For example, in any aspect or embodiment described herein, enzyme-linked immunosorbent assay (ELISA) (such as, titration ELISA), flowcytometry, surface plasmon resonance (SPR, such as Biacore™), bio-layer interferometry (BLI, such as Octet Red), or a combination thereof, is utilized in detecting and/or determining the binding capacity of an antibody. In any aspect or embodiment described herein, the binding affinity (Kd) of the antigen binding region or antibody, or antigen binding fragment thereof, to the target protein is less than about 100 μM, about 10 μM, about 1 μM, about 100 nM, about 10 nM, about 1 nM, about 100 pM, about 10 pM, or about 1 pM. In any aspect or embodiment described herein, the binding affinity (Kd) of the antibody or antigen binding fragment thereof to the target protein is about 1 pM to about 50 nM (e.g., about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 1 pM, about 2 pM, about 3 pM, about 4 pM, about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, any values in between, or a range from any combination of the values). In any aspect or embodiment described herein, the binding affinity (Kd) of the antigen binding region or antibody, or antigen binding fragment thereof, to the target protein is about 1 pM to about 15 nM (e.g., about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, any values in between, or a range from any combination of the values).

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

Any method for generating a heavy chain and/or a light chain that is capable of binding an epitope individually (e.g., the same epitope) can be used to produce the antibodies of the present disclosure (FnAbs or Shelly). In any aspect or embodiment described herein, the light chain, the heavy chain, the antigen binding region, the antibody binding arms (i.e., heavy chain and light chain), antibody or antigen-binding fragment or portion thereof, of the present disclosure are produced in vivo, in vitro (e.g., phage, yeast or ribosomal display), or any combination thereof.

In any aspect or embodiment described herein, the method further comprises an in vivo immunization method comprising: (a) immunizing an animal at least once with a modified peptide having an amino acid sequence of about 10 to about 20 amino acids identical to an amino acid sequence of the first epitope and/or the second epitope, except wherein one internal amino acid has been substituted with a non-native amino acid (nnAA); and (b) boosting the animal at least once with a first unmodified peptide comprising a core amino acid sequence identical to the modified peptide of step (a), except wherein the nnAA has been substituted with the native amino acid (nAA), a first N-terminal amino acid sequence that is not native to the protein of interest (e.g. antigen, the first antigen, and/or the second antigen), and a first C-terminal amino acid sequence that is not native to the protein of interest (e.g. antigen, the first antigen, and/or the second antigen).

In any aspect or embodiment described herein, the method further comprises: (c) cloning B-cells obtained from the animal; and (d) identifying a clone of step (c) that: (i) binds to the first unmodified peptide and a second unmodified peptide comprising a core amino acid sequence that is identical to the first unmodified peptide, a second N-terminal amino acid sequence that is not native to the protein of interest (e.g. antigen, the first antigen, and/or the second antigen), and a second C-terminal amino acid sequence that is not native to the protein of interest (e.g. antigen, the first antigen, and/or the second antigen); and (ii) does not bind to the modified peptide of step (a).

In any aspect or embodiment described herein, the method further comprises identifying a clone that binds to the protein of interest (e.g., antigen, the first antigen, and/or the second antigen).

In any aspect or embodiment described herein, the method further comprises: (c).cloning B-cells obtained from the animal; and (d) identifying a clone of step (c) that bind to the protein of interest (e.g., antigen, the first antigen, and/or the second antigen) in its native conformation.

In any aspect or method described herein, the method further comprises identifying a clone that binds to the protein of interest (e.g., antigen, the first antigen, and/or the second antigen).

In any aspect or embodiment described herein, the method further comprises an in vivo immunization method comprising immunizing an animal at least once with: (a) a first antigenic fusion peptide comprising a first antigenic peptide (M1) having an amino acid sequence of about 10 to about 20 amino acids identical to a first amino acid sequence (N1) of a protein of interest (e.g. antigen, the first antigen, and/or the second antigen) except wherein one internal amino acid has been substituted with a non-native amino acid (nnAA); and a second antigenic peptide (M2) having an amino acid sequence of about 10 to about 20 amino acids identical to a second amino acid sequence (N2) of a protein of interest (e.g. antigen, the first antigen, and/or the second antigen), wherein the first peptide and the second peptide are joined by a linker (A); and (b) boosting the animal at least once with a second antigenic fusion peptide comprising N1 joined to N2 by a linker (C), wherein the second antigenic fusion peptide further comprises a first N-terminal amino acid sequence (NT1) that is not native to the protein of interest (e.g. antigen, the first antigen, and/or the second antigen) and a first C-terminal amino acid sequence (CT1) that is not native to the protein of interest (e.g. antigen, the first antigen, and/or the second antigen).

In any aspect or embodiment described herein, the method further comprises an in vivo immunization method comprising immunizing an animal at least once with: (a) a first antigenic fusion peptide comprising a first antigenic peptide (M1) having an amino acid sequence of about 10 to about 20 amino acids identical to a first amino acid sequence (N1) of a protein of interest (e.g. antigen, the first antigen, and/or the second antigen) except wherein one internal amino acid has been substituted with a non-native amino acid (nnAA); and a second antigenic peptide (M2) having an amino acid sequence of about 10 to about 20 amino acids identical to a second amino acid sequence (N2) of a protein of interest (e.g. antigen, the first antigen, and/or the second antigen) except wherein one internal amino acid has been substituted with a non-native amino acid (nnAA), wherein the first antigenic peptide and the second antigenic peptide are joined by a linker (B); and (b) boosting the animal at least once with a second antigenic fusion peptide comprising N1 joined to N2 by a linker (C), wherein the second antigenic fusion peptide further comprises a first N-terminal amino acid sequence (NT1) that is not native to the protein of interest (e.g. antigen, the first antigen, and/or the second antigen) and a first C-terminal amino acid sequence (CT1) that is not native to the protein of interest (e.g. antigen, the first antigen, and/or the second antigen).

In any aspect or embodiment described herein, the method further comprises prior to step (b) immunizing the animal receiving the antigen of step (a)(i) at least once with a third antigenic fusion peptide comprising N1 joined to M2 by a linker (D).

In any aspect or embodiment described herein, the method further comprises: (c) cloning B-cells obtained from the animal; and (d) identifying a clone of step (c) that: (i) binds to a peptide comprising N1 joined to N2 by a linker (C), wherein the peptide further comprises a second N-terminal amino acid sequence (NT2) that is not native to the protein of interest (e.g. antigen, the first antigen, and/or the second antigen) and a second C-terminal amino acid sequence (CT2) that is not native to the protein of interest (e.g. antigen, the first antigen, and/or the second antigen); and (ii) does not bind to the first antigenic fusion peptide or the second antigenic fusion peptide.

In any aspect or embodiment described herein, generating the antigen binding region (e.g., antibody or an antigen-binding fragment or portion thereof) comprises: (a) immunizing an animal at least once with a modified peptide having an amino acid sequence of about 10 to about 20 amino acids identical to an amino acid sequence of the first epitope, the second epitope, or both the first epitope and the second epitope, depending on the aspect and/or embodiment, that includes the amino acids of the target sequence, except wherein one internal amino acid (e.g., the site of interest or adjacent to the site of interest) has been substituted with a non-native amino acid (nnAA); and (b) boosting the animal at least once with a first unmodified peptide comprising a core amino acid sequence identical to the modified peptide of step (a), except wherein the nnAA has been substituted with the native amino acid (nAA), a first N-terminal amino acid sequence that is not native to the first antigen, the second antigen, and/or the antigen, depending upon the aspect and/or embodiment, and a first C-terminal amino acid sequence that is not native to the first antigen, the second antigen, and/or the antigen, depending upon the aspect and/or embodiment. The method optionally, in any aspect or embodiment described herein, further comprising: (c) cloning B-cells obtained from the animal; and (d) identifying a clone of step (c) that: (i) binds to the first unmodified peptide and a second unmodified peptide comprising a core amino acid sequence that is identical to the first unmodified peptide, a second N-terminal amino acid sequence that is not native to the first antigen, the second antigen, and/or the antigen, depending upon the aspect and/or embodiment, and a second C-terminal amino acid sequence that is not native to the protein or protein of interest (e.g. antigen, the first antigen, and/or the second antigen); and (ii) does not bind to the modified peptide of step (a). The method optionally, in any aspect or embodiment described herein, further comprises: (c) cloning B-cells obtained from the animal; and (d) identifying a clone of step (c) that bind to the protein or peptide of interest (e.g., antigen, the first antigen, and/or the second antigen) in its native conformation.

In any aspect or embodiment described herein, the method further comprises identifying a clone that bind to the first epitope, the second epitope, or both the first epitope and the second epitope.

In any aspect or embodiment described herein, the first amino acid sequence of the protein of interest (e.g. antigen, the first antigen, and/or the second antigen) and the second amino acid sequence of the protein of interest (e.g. antigen, the first antigen, and/or the second antigen) are from the same protein (i.e., the protein of interest of the first amino acid sequence and the protein of interest of the second amino acid sequence are the same protein).

In any aspect or embodiment described herein, the first amino acid sequence of the protein of interest (e.g. antigen, the first antigen, and/or the second antigen) and the second amino acid sequence of the protein of interest (e.g. antigen, the first antigen, and/or the second antigen) are from different proteins (i.e., the protein of interest of the first amino acid sequence and the protein of interest of the second amino acid sequence are different proteins).

In any aspect or embodiment described herein, the animal is a human, a rabbit, a mouse, a rat, a goat, a cow, a pig, a camelid, or a chicken.

In any aspect or embodiment described herein, the animal is a non-human animal that has a human or humanized immune system.

In any aspect or embodiment described herein, the peptide is administered with an adjuvant. In any aspect or method described herein, the modified peptide, the first unmodified peptide, the second unmodified peptide, or a combination thereof, is administered with an adjuvant.

In any aspect or embodiment described herein, the adjuvant is Complete Freund's Adjuvant (CFA), Incomplete Freund's Adjuvant (IFA), aluminum, monophosphoryl lipid A (MPL) and aluminum salt (AS04), oil-in-water emulsion, oil-in-water emulsion of squalene (MF59), AS03 (Vitamin E, Surfactant polysorbate 80, and squalene), MPL and QS-21 in a liposome formulation (AS01), or cytosine phosphoguanine (CpG).

In any aspect or embodiment described herein,

In any aspect or embodiment described herein, the modified peptide and/or the first unmodified peptide is conjugated to one or more carriers.

In any aspect or embodiment described herein, the first antigenic fusion peptide is conjugated to one or more carriers.

In any aspect or embodiment described herein, the carrier is keyhole limpet hemocyanin (KLH) or ovalbumin.

In any aspect or embodiment described herein, the method further comprises preparing/producing a site directed binding agent to the first epitope and/or the second epitope, the method comprising: (a) providing a modified peptide having an amino acid sequence of about 10 to about 20 amino acids identical to an amino acid sequence of the first epitope and/or the second epitope (e.g. epitopes on the antigen, the first antigen, and/or the second antigen) except wherein one internal amino acid has been substituted with a non-native amino acid (nnAA); (b) screening the modified peptide against a library; (c) isolating one or more binding agents that bind to the modified peptide; (d) generating a library of clonotypes of the one or more binding agents isolated in step (c); (e) screening the library of clonotypes against: (i) the modified peptide of step (a); and (ii) a first unmodified peptide comprising a core amino acid sequence identical to the modified peptide of step (a), except wherein the nnAA has been substituted with the native amino acid (nAA), a first N-terminal amino acid sequence that is not native to the first antigen, the second antigen, and/or the antigen, depending upon the aspect and/or embodiment, and a first C-terminal amino acid sequence that is not native to the first antigen, the second antigen, and/or the antigen, depending upon the aspect and/or embodiment; and (f) isolating a binding agent that bind to both the first unmodified peptide and the modified peptide.

In any aspect or embodiment described herein, the method further comprising: (g) generating a library of clonotypes of the binding agent isolated in step (f); (h) screening the library of clonotypes of step (g) against: (i) the modified peptide; (ii) the first unmodified peptide; and (iii) a second unmodified peptide comprising a core amino acid sequence that is identical to the first unmodified peptide, a second N-terminal amino acid sequence that is not native to the protein of interest (e.g. antigen, the first antigen, and/or the second antigen), and a second C-terminal amino acid sequence that is not native to the protein of interest (e.g. antigen, the first antigen, and/or the second antigen); and (i) isolating a binding agent that binds to the first unmodified peptide and the second unmodified peptide, wherein the binding agent does not bind to the modified peptide.

In any aspect or embodiment described herein, the method further comprises preparing/producing a site directed conformational binding agent to the first epitope and/or the second epitope (e.g. epitope on antigen, the first antigen, and/or the second antigen), the method comprising: (a) providing a first antigenic fusion peptide comprising a first antigenic peptide (M1) having an amino acid sequence of about 10 to about 20 amino acids identical to a first amino acid sequence (N1) of a protein of interest (e.g. antigen, the first antigen, and/or the second antigen) except wherein one internal amino acid has been substituted with a non-native amino acid (nnAA); and a second antigenic peptide (M2) having an amino acid sequence of about 10 to about 20 amino acids identical to a second amino acid sequence (N2) of a protein of interest (e.g. antigen, the first antigen, and/or the second antigen), wherein the first antigenic peptide and the second antigenic peptide are joined by a linker (A); (b) screening M1 against a library; (c) isolating one or more binding agents that bind to M1; (d) generating a library of clonotypes of the one or more binding agents isolated in step (c); (e) screening the library of clonotypes against: (i) M1; and (ii) a first peptide comprising N1 joined to N2 by a linker (B), wherein the first peptides further comprises a first N-terminal amino acid sequence (NT1) that is not native to the protein of interest (e.g. antigen, the first antigen, and/or the second antigen) and a first C-terminal amino acid sequence (CT1) that is not native to the protein of interest (e.g. antigen, the first antigen, and/or the second antigen); and (f) isolating a binding agent that bind to M1 and the first peptide.

In any aspect or embodiment described herein, the method further comprises: (g) generating a library of clonotypes of the binding agents isolated in step (f); (h) screening the library of clonotypes of step (g) against: (i) M1; (ii) the first peptide; and (iii) a second peptide comprising N1 joined to N2 by a linker (D), wherein the second peptide further comprises a second N-terminal amino acid sequence (NT2) that is not native to the protein of interest (e.g. antigen, the first antigen, and/or the second antigen) and a second C-terminal amino acid sequence (CT2) that is not native to the protein of interest (e.g. antigen, the first antigen, and/or the second antigen); (i) isolating a binding agent that bind to: (i) M1, the first peptide, and the second peptide; or (ii) the first peptide and the second peptide.

In any aspect or method described herein, generating the antigen binding region (e.g., antibody or an antigen-binding fragment or portion thereof) comprises: (a) providing a modified peptide having an amino acid sequence of about 10 to about 20 amino acids identical to an amino acid sequence of the first epitope and/or the second epitope and includes the amino acids of the target sequence, except wherein the wild-type amino acid at the site of interest (e.g., a variant site) has been substituted with a non-native amino acid (nnAA); and (b) screening the modified peptides against a library; (c) isolating one or more binding agents that bind to the modified peptide; (d) generating a library of clonotypes of the one or more binding agents isolated in step (c); (e) screening the library of clonotypes against: (i) the modified peptide of step (a); and (ii) a first unmodified peptide comprising a core amino acid sequence identical to the modified peptide of step (a), except wherein the non-native amino acid (nnAA) has been substituted with the wild-type amino acid (nAA) and comprising a first N-terminal amino acid sequence that is not native to the first antigen, the second antigen, and/or the antigen, depending upon the aspect and/or embodiment and a first C-terminal amino acid sequence that is not native to the first antigen, the second antigen, and/or the antigen, depending upon the aspect and/or embodiment; (f) isolating a binding agent that bind to both the first unmodified peptide and the modified peptide, wherein the binding agent is the antigen binding region (e.g., antibody or an antigen-binding fragment or portion thereof) or derived therefrom.

In any aspect or method described herein, generating the antigen binding region (e.g., antibody or an antigen-binding fragment or portion thereof) comprises: (a) providing a modified peptide having an amino acid sequence of about 10 to about 20 amino acids identical to an amino acid sequence of the first epitope and/or the second epitope and includes the amino acids of the target sequence, except wherein the wild-type amino acid at the site of interest (e.g., a variant site) has been substituted with a non-native amino acid (nnAA); (b) screening the modified peptides against a library; (c) isolating one or more binding agents that bind to the modified peptide; (d) generating a library of clonotypes of the one or more binding agents isolated in step (c); (e) screening the library of clonotypes against: (i) the modified peptide of step (a); and (ii) a first unmodified peptide comprising a core amino acid sequence identical to the modified peptide of step (a), except wherein the non-native amino acid (nnAA) has been substituted with the wild-type amino acid (nAA) and comprising a first N-terminal amino acid sequence that is not native to the first antigen, the second antigen, and/or the antigen, depending upon the aspect and/or embodiment, and a first C-terminal amino acid sequence that is not native to the first antigen, the second antigen, and/or the antigen, depending upon the aspect and/or embodiment; (f) isolating a binding agent that bind to both the first unmodified peptide and the modified peptide; (g) generating a library of clonotypes of the binding agent isolated in step (f); (h) screening the library of clonotypes of step (g) against: (i) the modified peptide; (ii) the first unmodified peptide; and (iii) a second unmodified peptide comprising a core amino acid sequence that is identical to the first unmodified peptide, a second N-terminal amino acid sequence that is not native to the first antigen, the second antigen, and/or the antigen, depending upon the aspect and/or embodiment, and a second C-terminal amino acid sequence that is not native to the first antigen, the second antigen, and/or the antigen, depending upon the aspect and/or embodiment; and (f) isolating a binding agent that binds to the first unmodified peptide and the second unmodified peptide, wherein the binding agent does not bind the modified peptide, and the binding agent is the antigen binding region (e.g., antibody or an antigen-binding fragment or portion thereof) or derived therefrom.

In any aspect or method described herein, generating the antigen binding region (e.g., antibody or an antigen-binding fragment or portion thereof) comprises: (a) providing a modified peptide having an amino acid sequence of about 10 to about 20 amino acids identical to an amino acid sequence of the first epitope and/or the second epitope and includes the amino acids of the target sequence, except wherein the wild-type amino acid at the site of interest (e.g., a variant site) has been substituted with a non-native amino acid (nnAA); (b) screening the modified peptides against a library; (c) isolating one or more binding agents that bind to the modified peptide; (d) generating a library of clonotypes of the one or more binding agents isolated in step (c); I screening the library of clonotypes against: (1) the modified peptide of step (a); and (2) a first unmodified peptide comprising a core amino acid sequence identical to the modified peptide of step (a), except wherein the non-native amino acid (nnAA) has been substituted with the wild-type amino acid (nAA) and comprising a first N-terminal amino acid sequence that is not native to the protein of interest (e.g. antigen, the first antigen, and/or the second antigen) and a first C-terminal amino acid sequence that is not native to the protein of interest (e.g. antigen, the first antigen, and/or the second antigen); (f) isolating a binding agent that bind to both the first unmodified peptide and the modified peptide; (g) generating a library of clonotypes of the binding agent isolated in step (f); (h) screening the library of clonotypes of step (g) against: (1) the modified peptide; (2) the first unmodified peptide; and (3) a second unmodified peptide comprising a core amino acid sequence that is identical to the first unmodified peptide, except wherein the wild-type amino acid (nAA) has been substituted with an amino acid corresponding to a variant and comprising a second N-terminal amino acid sequence that is not native to the first antigen, the second antigen, and/or the antigen, depending upon the aspect and/or embodiment, and a second C-terminal amino acid sequence that is not native to the first antigen, the second antigen, and/or the antigen, depending upon the aspect and/or embodiment; and (i) isolating a binding agent that binds to the second unmodified peptide and does not bind the first modified peptide, wherein the binding agent is the antigen binding region (e.g., antibody or an antigen-binding fragment or portion thereof) or derived therefrom. In any aspect or method described herein, the library is an antibody display library, and the binding agent is an antibody or an antigen-binding fragment or portion thereof. In any aspect or embodiment described herein, the display library is an aptamer display library and the binding agent is an aptamer.

In any aspect or method described herein, the non-native amino acid (nnAA) is offset by +/−4, +/−3, +/−2, or +/−1 relative to the site of interest (position 0). In any aspect or method described herein, the non-native amino acid (nnAA) is offset by +/−1, +/−2, or +/−3 relative to the site of interest when it is a post-translational modification site (position 0). In any aspect or method described herein, the non-native amino acid (nnAA) is offset by +/−2 position relative to the site of interest when it is a splice site/junction (position 0)).

In any aspect or embodiment described herein, the non-native amino acid (nnAA) is a non-synonymous amino acid.

In any aspect or embodiment described herein, the non-native amino acid (nnAA) is phosphorylated, acetylated, isocyanated, sulfated, or nitrated.

In any aspect or embodiment described herein, the non-native amino acid (nnAA) is O-phosphoserine (SEP).

In any aspect or embodiment described herein, the non-native amino acid (nnAA) is phosphotyrosine.

In any aspect or embodiment described herein, the non-native amino acid (nnAA) is phosphothreonine.

In any aspect or embodiment described herein, the N-terminal amino acid sequence is

SerGlySer,
GlySerGly,
GlyGlyGly,
or
SerSerSer.

In any aspect or embodiment described herein, the C-terminal amino acid sequence is

SerGlySer,
GlySerGly,
GlyGlyGly,
or
SerSerSer.

### Methods of Using Antigen Binding Regions or Antibodies (or Antigen-Binding Fragments or Portions Thereof) of the Present Disclosure

It would be readily apparent to one skilled in the art that the antigen binding region of any the present disclosure, the chimeric antibody or fragment thereof of the present disclosure, the chimeric antibody of the present disclosure, or the pharmaceutical composition of the present disclosure, produced by the methods described herein, are useful in a variety of diagnostic and therapeutic applications. For example, the antigen binding region of any the present disclosure, the chimeric antibody or fragment thereof of the present disclosure, the chimeric antibody of the present disclosure, or the pharmaceutical composition of the present disclosure, are useful in the development of vaccines, diagnostics, biosimilars, chimeric antigen receptor T-cell (CAR-T) therapies, therapeutics, and multi-specific antibodies.

An additional aspect of the present disclosure relates to a method of treating a disease or disorder in a subject in need thereof comprising administering the antigen binding region of any the present disclosure, the chimeric antibody or fragment thereof of the present disclosure, the chimeric antibody of the present disclosure, or the pharmaceutical composition of the present disclosure.

A further aspect of the present disclosure provides a method of treating, preventing, and/or ameliorating at least one symptom of a disease or disorder (e.g., pneumonia, a viral infection, a bacterial infection, a coronavirus infection (such as, severe acute respiratory syndrome coronavirus 2 (SARS-CoV-2) or Coronavirus Disease 2019 (COVID-19)), a Human-Immunodeficiency Virus (HIV) infection, an ebolavirus infection (such as Ebola virus (EBOV) infection, Sudan virus (SUDV) infection, Bundibugyo virus (BDBV) infection, Reston virus (RESTV) infection, Tai Forest virus (TAFV) infection), etc.) in a subject in need thereof. The method comprises: providing a subject in need thereof; and administering an effective amount of the antigen binding region of any the present disclosure, the chimeric antibody or fragment thereof of the present disclosure, the chimeric antibody of the present disclosure, the pharmaceutical composition of the present disclosure, and/or the formulation of the present disclosure, wherein the antigen binding region, chimeric antibody or fragment thereof of the present disclosure, chimeric antibody, pharmaceutical composition, and/or formulation of the present disclosure effectuates the prevention, treatment, or amelioration of at least one symptom of the disease or disorder.

An additional aspect of the present disclosure provides, pharmaceutical compositions or formulations comprising the antigen binding region of the present disclosure, an antibody of the present disclosure, or an antigen binding fragment of the present disclosure. In any aspect or embodiment described herein the pharmaceutical composition(s) or formulation(s) of the present disclosure further comprises an effective amount of an excipient (e.g., an effective amount of a pharmaceutically acceptable excipient) or carrier (e.g., an effective amount of a pharmaceutically acceptable carrier). As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the antigen binding region of any the present disclosure, the chimeric antibody or fragment thereof of the present disclosure and the chimeric antibody of the present disclosure, use thereof in the compositions is contemplated. Supplementary active compounds can also be incorporated into the compositions.

The description provides methods for preparing pharmaceutical compositions or formulations. Such methods comprise formulating an effective amount of a pharmaceutically acceptable carrier or excipient with one or more antibody as described herein. Such compositions or formulations can further include additional active agents as described above. Thus, the present disclosure further describes methods for preparing a pharmaceutical composition or formulation.

A pharmaceutical composition or formulation of the present disclosure is formulated to be compatible with its intended route of administration. Examples of routes of administration include parenteral (e.g., intravenous, intradermal, subcutaneous, intramuscular, intraperitoneal, intranodal, and intrasplenic) administration. Solutions or suspensions used for parenteral, intradermal, or subcutaneous application can include the following components: a sterile diluent (such as water for injection, saline solution, fixed oils, polyethylene glycols, glycerin, propylene glycol, or other synthetic solvents); antibacterial agents (such as benzyl alcohol); antioxidants (such as ascorbic acid or sodium bisulfate); chelating agents (such as ethylenediamine-tetraacetic acid); buffers (such as acetates, citrates or phosphates); and agents for the adjustment of tonicity (such as sodium chloride or dextrubinrubi). pH can be adjusted with acids or bases, such as hydrochloric acid or sodium hydroxide. The parenteral preparation can be enclosed in ampules, disposable syringes or multiple dose vials made of glass or plastic.

Pharmaceutical compositions or formulations suitable for injectable use, in any aspect or embodiment described herein, include sterile aqueous solutions (where water soluble) or dispersions and sterile powders for the extemporaneous preparation of sterile injectable solutions or dispersions. In any aspect or embodiment described herein, for intravenous administration, suitable carriers include physiological saline, bacteriostatic water, Cremophor E L (BASF; Parsippany, N.J.) or phosphate buffered saline (PBS). In all cases, the composition or formulation must be sterile and should be fluid to the extent that easy syringability exists. It must be stable under the conditions of manufacture and storage and must be preserved against the contaminating action of microorganisms such as bacteria and fungi. In any aspect or embodiment described herein, the carrier can be a solvent or dispersion medium containing, for example, water, ethanol, polyol (for example, glycerol, propylene glycol, and liquid polyethylene glycol, and the like), and suitable mixtures thereof. In any aspect or embodiment described therein, the proper fluidity can be maintained, for example, by the use of a coating (such as lecithin), by the maintenance of the required particle size in the case of dispersion, by the use of surfactants, or a combination thereof. In any aspect or embodiment described herein, prevention of the action of microorganisms is achieved by various antibacterial and antifungal agents (for example, chlorobutanol, phenol, ascorbic acid, and the like). In any aspect or embodiment described herein, it will be preferable to include isotonic agents (for example, sugars, polyalcohols (such as mannitol or sorbitol), or sodium chloride) in the composition or formulation. In any aspect or embodiment described herein, prolonged absorption of the injectable compositions or formulations can be brought about by including in the composition or formulation an agent which delays absorption (for example, aluminum monostearate, gelatin, or a combination thereof).

Sterile injectable solutions can be prepared by incorporating the chimeric antibody in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate parenteral compositions or formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by and directly dependent on the unique characteristics of the antigen binding region of any the present disclosure, the chimeric antibody or fragment thereof of the present disclosure and the chimeric antibody of the present disclosure, and the particular therapeutic effect to be achieved, and the limitations inherent in the art of compounding such an antibody for the treatment of subjects.

In any aspect or embodiment described herein, the method can further include the step of administering (e.g., to a human) a dosage from about 100 ng to about 200 mg of a therapeutic or pharmaceutical composition or formulation as described herein. In any aspect or embodiment described herein, the pharmaceutical composition or formulation as described herein may contain mannitol as carrier, and the composition or formulation is administered from 10 μg to 200 mg, preferably 20 to 100 mg, in a single administration (e.g., to a human).

In any aspect or embodiment described herein, preferred pharmaceutically acceptable carriers can comprise, for example, xanthan gum, locust bean gum, galactose, other saccharides, oligosaccharides, polysaccharides, starch, starch fragments, dextrins, British gum, or mixtures thereof. Advantageously, in any aspect or embodiment described herein, the pharmaceutically acceptable carrier is of natural origin. In any aspect or embodiment described herein, the pharmaceutically acceptable carrier is, or further comprise, an inert saccharide diluent selected from a monosaccharide or disaccharide (e.g., mannitol).

In any aspect or embodiment described herein, the composition further comprises at least one stabilizer (e.g., one or more of salt(s), saccharide(s), and/or amino acid(s)). In any aspect or embodiment described herein, the composition further comprises at least one surfactant. In any aspect or embodiment described herein, the composition further comprises at least one buffering agent.

The term “surfactant” as used herein refers to a pharmaceutically acceptable, surface-active agent. For example, in any aspect or embodiment described herein, a non-ionic surfactant is used. Examples of pharmaceutically acceptable surfactants include, but are not limited to, polyoxyethylen-sorbitan fatty acid esters (Tween®), polyoxyethylene alkyl ethers (Brij), alkylphenylpolyoxyethylene ethers (Triton X), polyoxyethylenepolyoxypropylene copolymers (Poloxamer, Pluronic), sodium dodecyl sulphate (SDS), or mixture thereof. In any aspect or embodiment described herein, the polyoxyethylene-sorbitan fatty acid esters include or are polysorbate 20 (polyoxyethylene sorbitan monolaureate, sold under the trademark Tween 20™) and/or polysorbate 80 (polyoxyethylene sorbitan monooleate, sold under the trademark Tween 80™). In any aspect or embodiment described herein, the polyethylene-polypropylene copolymers include or are Pluronic® F68 and/or Poloxamer188™. In any aspect or embodiment described herein, the polyoxyethylene alkyl ethers includes or are those sold under the trademark Brij™. In any aspect or embodiment described herein, the alkylphenylpolyoxyethylene ethers includes or are Triton X and/or p-tert-octylphenoxy polyethoxyethanol (such as, Triton X-100™). In any aspect or embodiment described herein, the surfactants include or are polyoxyethylene-sorbitan fatty acid esters, such as polysorbate 20 or polysorbate 80. In any aspect or embodiment described herein, the surfactant is Poloxamer 188™.

The term “buffering agent” as used herein refers to a pharmaceutically acceptable excipient, which stabilizes the pH of a pharmaceutical preparation. Suitable buffers are well known in the art and can be found in the literature. In any aspect or embodiment described herein, pharmaceutically acceptable buffers comprise, but are not limited to histidine-buffers, citrate-buffers, succinate-buffers, acetate-buffers, arginine-buffers, phosphate-buffers, or mixtures thereof. Buffering agents are thus histidine salts, citrate salts, succinate salts, acetate salts, malate salts, phosphate salts and lactate salts. Buffering agents of particular interest comprise L-histidine or mixtures of L-histidine and L-histidine hydrochloride or L-histidine acetate with pH adjustment with an acid or a base known in the art. In any aspect or embodiment described herein, the above-mentioned buffers are used in an amount of about 5 mM to about 100 mM, particularly of about 10 mM to about 30 mM, and more particularly of about 20 mM. Independently from the buffer used, in any aspect or embodiment describe herein, the pH can be adjusted to a value in the range from about 4.5 to about 7.0, and particularly to a value in the range from about 5.0 to about 6.0, and most particularly to pH 6.0.+−0.0.03 with an acid or a base known in the art, e.g. hydrochloric acid, acetic acid, phosphoric acid, sulfuric acid and citric acid, sodium hydroxide and potassium hydroxide.

The term “stabilizer” as used herein refers to a pharmaceutical acceptable excipient, which protects the active pharmaceutical ingredient and/or the formulation from chemical and/or physical degradation during manufacturing, storage and application. In any aspect or embodiment described herein, stabilizers include, but are not limited to, saccharides, amino acids, polyols (e.g. mannitol, sorbitol, xylitol, dextran, glycerol, arabitol, propylene glycol, polyethylene glycol), cyclodextrines (e.g. hydroxypropyl-β-cyclodextrine, sulfobutyl-ethyl-β-cyclodextrine, .beta.-cyclodextrine), polyethylenglycols (e.g. PEG 3000, PEG 3350, PEG 4000, PEG 6000), albumines (human serum albumin (HSA), bovine serum albumin (BSA)), salts (e.g. sodium chloride (saline), magnesium chloride, calcium chloride), and/or chelators (e.g. EDTA). In any aspect or embodiment described herein, the stabilizer is selected from the group consisting of saccharides, polyols, and amino acids. In any aspect or embodiment described herein, the one or more stabilizers is present in the formulation in an amount of about 10 mM to about 500 mM, particularly in an amount of about 140 to about 250 mM, and more particularly in an amount of about 210 mM to about 240 mM. For example, in any aspect or embodiment described herein, sucrose or trehalose are used as stabilizers in an amount of about 220 mM to about 240 mM.

The term “saccharide” as used herein includes monosaccharides and oligosaccharides. A monosaccharide is a monomeric carbohydrate which is not hydrolysable by acids, including simple sugars and their derivatives, e.g. aminosugars. Saccharides are usually in their D conformation. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a linear chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. Preferred saccharides for use in the present disclosure are sucrose and trehalose (i.e., α,α-D-trehalose), most preferred is sucrose. Trehalose is available as trehalose dihydrate. In any aspect or embodiment described herein, the at least one saccharide(s) can be present in the formulation in an amount of about 10 to about 500 mM, preferably in an amount of about 200 to about 300 mM, more preferably in an amount of about 220 to about 250 mM, particularly an amount of about 220 mM or about 240 mM, most preferably in an amount of about 220 mM.

The term “amino acid” as used herein as a stabilizer refers to a pharmaceutically acceptable organic molecule possessing an amino moiety located at a-position to a carboxylic group. In any aspect or embodiment described herein, the amino acid is one or more of arginine, glycine, ornithine, lysine, histidine, glutamic acid, asparagic acid, isoleucine, leucine, alanine, phenylalanine, tyrosine, tryptophane, methionine, serine, and proline. In any aspect or embodiment described herein, the amino acid employed is the L-form. Basic amino acids, such as arginine, histidine, or lysine, are preferably employed in the form of their inorganic salts (advantageously in the form of the hydrochloric acid salts, i.e., as amino acid hydrochlorides). In any aspect or embodiment descried herein, the amino acid is methionine. In any aspect or embodiment described herein, the amino acid (such as methionine) is used at a concentration of about 5 to about 25 mM or about 10 mM.

In any aspect or embodiment described herein, the stabilizer includes or is one or more lyoprotectant. The term “lyoprotectant” as used herein refers to a pharmaceutically acceptable excipients, which protect the labile active ingredient (e.g., a protein) against destabilizing conditions during the lyophilisation process, subsequent storage and reconstitution. In any aspect or embodiment described herein, the lyoprotectants comprise, but are not limited to, the group consisting of saccharides, polyols (such as e.g., sugar alcohols), and amino acids. In any aspect or embodiment described herein, the one or more lyoprotectant is selected from the group consisting of saccharides such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine, or mixtures thereof. In any aspect or embodiment described herein, the one or more lyoprotectant is used in an amount of about 10 to 500 mM, preferably in an amount of about 10 to about 300 mM and more preferably in an amount of about 100 to about 300 mM.

In any aspect or embodiment described herein, the stabilizer includes or is one or more antioxidant. The term “antioxidant” as used herein refers to a pharmaceutically acceptable excipient, which prevent oxidation of the active pharmaceutical ingredient. In any aspect or embodiment described herein, the one or more antioxidant comprises, but are not limited to, ascorbic acid, glutathione, cysteine, methionine, citric acid, and EDTA. In any aspect or embodiment described herein, the one or more antioxidant is used in an amount of about 0.01 to about 100 mM, preferably in an amount of about 5 to about 50 mM and more preferably in an amount of about 5 to about 25 mM.

In any aspect or embodiment described herein, the compositions or formulations described herein further comprise one or more tonicity agents. The term “tonicity agents” as used herein refers to pharmaceutically acceptable excipients used to modulate the tonicity of the formulation. The formulation can be hypotonic, isotonic or hypertonic. Isotonicity in general relates to the osmotic pressure of a solution, usually relative to that of human blood serum (around 250-350 mOsmol/kg). The formulation according to the present disclosure can be hypotonic, isotonic or hypertonic, but will preferably be isotonic. In any aspect or embodiment described herein, an isotonic formulation is liquid or liquid reconstituted from a solid form (e.g., from a lyophilized form), and denotes a solution having the same tonicity as some other solution with which it is compared, such as physiologic salt solution and the blood serum. In any aspect or embodiment described herein, the one or more tonicity agents includes or is selected from sodium chloride, potassium chloride, glycerine and any component from the group of amino acids or sugars, in particular glucose. In any aspect or embodiment described herein, the one or more tonicity agents is used in an amount of about 5 mM to about 500 mM. Within the stabilizers and tonicity agents there is a group of compounds which can function in both ways, i.e., they can at the same time be a stabilizer and a tonicity agent. Examples thereof can be found in the group of sugars, amino acids, polyols, cyclodextrines, polyethyleneglycols and salts. An example of a sugar which can at the same time be a stabilizer and a tonicity agent is trehalose.

The term “polyols” as used herein denotes pharmaceutically acceptable alcohols with more than one hydroxy group. In any aspect or embodiment described herein, the one or more polyols is selected from mannitol, sorbitol, glycerine, dextran, glycerol, arabitol, propylene glycol, polyethylene glycol, and combinations thereof. In any aspect or embodiment described herein, the one or more polyols is used in an amount of about 10 mM to about 500 mM, particularly in an amount of about 10 to about 250 mM and more particularly in an amount of about 200 to about 250 mM.

In any aspect or embodiment described herein, the composition or formulations described herein may further includes adjuvants (such as preservatives, wetting agents, emulsifying agents and dispersing agents). Prevention of presence of microorganisms may be ensured both by sterilization procedures, and by the inclusion of various antibacterial and antifungal agents, e.g., paraben, chlorobutanol, phenol, sorbic acid, and the like. In any aspect or embodiment described herein, the one or more preservative is used in an amount of about 0.001 to about 2% (w/v). In any aspect or embodiment described herein, the one or more preservative is selected from ethanol, benzyl alcohol, phenol, m-cresol, p-chlor-m-cresol, methyl or propyl parabens, benzalkonium chloride.

Advantageously, the present disclosure relates to a pharmaceutical composition or formulation as defined above, which is in the form of a liposome, or nano particles, or in the form of a solution. An advantageous solution is a solution comprising from 1 to 15%, in particular about 10% of mannitol. The solution should be iso-osmolar.

### Definitions

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. For example, in any aspect or embodiment described herein “antibody” as used herein in the specification and claims refers to a protein or immunoglobulin comprising at least two heavy chains (H chains) and two light chains (L chains) connected or stabilized by disulfide bonds. Each L chain is linked to an H chain by one covalent disulfide bond, while the two H chains are linked to each other by one or more disulfide bonds depending on the H chain isotype. Each H and L chain also has regularly spaced intrachain disulfide bridges. For the structure and properties of the different classes of antibodies, see, e.g., Basic and Clinical Immunology. 8th edition, Daniel P. Stites, Abba I. Terr and Tristram G. Parslow (eds.), Appleton & Lange, Norwalk, Conn., 1994, page 71 and Chapter 6.

Each heavy chain is comprised of, at the N-terminus, a heavy chain variable region (V_{H} or HVR) and a heavy chain constant region (C_{H} or HCR). The heavy chain-constant region comprises three heavy chain-constant domains (C_{H1}, C_{H2}, and C_{H3}) or four heavy chain-constant domains (IgM-type or IgE-type antibodies; C_{H1}, C_{H2}, C_{H3} and C_{H4}) wherein the first constant domain C_{H1} is adjacent to the variable region and may be connected to the second constant domain C_{H2} by a hinge region. Each light chain is comprised of, at the N-terminus, a light chain variable region (V_{L} or LVR) and a light chain constant region (C_{L} or LCR). The light chain-constant region consists only of one constant domain. The V_{L} is aligned with the V_{H} and the C_{L} is aligned with the first constant domain of the heavy chain (C_{H1}). Particular amino acid residues are believed to form an interface between the light chain and heavy chain variable domains. The pairing of a V_{H} and V_{L} together forms a single antigen-binding site.

The term “variable” refers to the fact that certain segments of the variable domains differ extensively in sequence among antibodies. The variable (V) domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids, which are more conserved, separated by shorter regions of extreme variability called “hypervariable regions” or complementary determining regions (CDRs) that are each 9-12 amino acids long. Native heavy and light chain variable regions/domains comprise three CDRs (CDR1, CDR2, and CDR3) and four FRs (FR1, FR2, FR3, and FR4) that largely adopting a beta-sheet configuration, connecting the three hypervariable regions (CDRs), arranged from amino-terminus to carboxy-terminus in the following order: FR1, CDR1, FR2, CDR2, FR3, CDR3, FR4. The hypervariable regions in each chain are held together in close proximity by the FRs, wherein the hypervariable regions from the other chain, can contribute to the formation of the antigen-binding site of the antibody (see Kabat et al., Sequences of Proteins of Immunological Interest. 5th Ed. Public Health Service, National Institutes of Health, Bethesda, Md. (1991)) or fragment/derivative thereof.

The term “chimeric antibody” as used herein refers to antibodies, bispecific antibodies, and multispecific antibodies wherein the heavy chain variable region and the light chain variable region independently bind an epitope (e.g., the same epitope, different epitopes on the same antigen, or epitopes on different antigens).

The term “hypervariable region” when used herein refers to the amino acid residues of an antibody that are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about residues 26-35 (H1), 50-65 (H2) and 95-102 (H3) in the V_{H} (in one embodiment, H1 is around about residues 31-35); Kabat et al., Sequences of Proteins of Immunological Interest. 5th Ed. Public Health Service, National Institutes of Health, Bethesda, Md. (1991)) and/or those residues from a “hypervariable loop” (e.g., residues 26-32 (L1), 50-52 (L2), and 91-96 (L3) in the V L, and 26-32 (H1), 53-55 (H2), and 96-101 (H3) in the V H; Chothia and Lesk, J. Mol. Biol. 196:901-917 (1987)).

The Kabat numbering system is generally used when referring to a residue in the variable domain (approximately residues 1-107 of the light chain and residues 1-113 of the heavy chain) (e.g., Kabat_{et al}., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

The constant regions (CRs) of the antibodies are not involved directly in binding an antibody to an antigen, but can mediate the binding of the immunoglobulin to host tissues or factors, including various cells of the immune system (e.g., effector cells) and the first component (C1q) of the classical complement system. The variable regions of the heavy and light chains contain a binding domain that interacts with an antigen. The heavy chain constant regions may be of any type, such as γ-, δ-, α-, μ- or ε-type heavy chains. The five classes or isotypes of immunoglobulins (IgA, IgD, IgE, IgG, and IgM) are classified based on the amino acid sequences of the constant domains of their heavy chains, wherein the heavy chains are respectively designated α, δ, γ, ε, and μ. The γ and α classes are further divided into subclasses on the basis of relatively minor differences in C H sequence and function, e.g., humans express the following subclasses: IgG1, IgG2, IgG3, IgG4, IgA1, and IgA2. In any aspect or embodiment described herein, the heavy chain constant region of the antibody is a γ-chain. Furthermore, the light chain constant region may also be of any type, such as x-type light chain or k-type light chain, which is based on the amino acid sequence of their constant domains. In any aspect or embodiment, the light chain constant region of the antibody is a κ-chain.

In any aspect or embodiment described herein, the term antibody refers to a population of antibodies of the same kind (e.g., all antibodies of the population exhibit the features used for defining the antibody). In any aspect or embodiment described herein, all antibodies in the population have the same amino acid sequence.

An “intact” antibody is one which comprises an antigen-binding site as well as a C_{L} and at least heavy chain constant domains, C_{H1}, C_{H2}, and C_{H3}. The constant domains can be native sequence constant domains (e.g., human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “monoclonal antibody” as used herein refers to an antibody obtained from a population of substantially homogeneous antibodies, i.e., the individual antibodies comprising the population are identical except for possible mutations (e.g., naturally occurring mutations) that may be present in minor amounts. Thus, the modifier “monoclonal” indicates the character of the antibody as not being a mixture of discrete antibodies. In any aspect or embodiment described herein, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a bispecific antibody, a multispecific antibody, etc., and that an antibody comprising the altered target binding sequence is also a monoclonal antibody of the present disclosure. In contrast to polyclonal antibody preparations, which typically include different antibodies directed against different determinants (epitopes), each monoclonal antibody of a monoclonal antibody preparation is directed against the same determinant (epitope) or determinants (epitopes)—i.e., they have the same specificity. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, such as from a single clone of a B lymphocyte or by a cell (e.g., HEK293 cell) engineered (e.g., the light and heavy chain genes of a single antibody have been transfected into the cell) to express a single antibody, and is not to be construed as requiring production of the antibody by any particular method. For example, the monoclonal antibodies to be used in accordance with the present disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest (e.g., antigen, the first antigen, and/or the second antigen).

The term “humanized antibody” as used herein in the specification and claims refers to an antibody from non-human species whose protein sequences have been modified to increase similarity to antibody variants produced naturally in humans, such as an amino acid sequence characteristic of an antibody derived from a non-human has replaced a corresponding position of a human antibody. Thus, in any aspect or embodiment described herein, humanized forms of non-human (e.g., murine) antibodies are chimeric antibodies that contain minimal sequence derived from non-human immunoglobulin. In any aspect or embodiment described herein, a humanized antibody is a human immunoglobulin (recipient antibody) in which residues from a V_{H} or HVR of the recipient are replaced by residues from a V_{H} or HVR of a non-human species (donor antibody), such as mouse, rat, rabbit, or nonhuman primate having the desired specificity, affinity, and/or capacity. Examples of the humanized antibody include an antibody having heavy chain CDR1 to CDR3 and light chain CDR1 to CDR3 derived from an antibody prepared from a non-human antibody and in which all other regions comprising respective four framework regions (FRs) of the heavy chain and the light chain are derived from a human antibody. Such an antibody may be referred to as a CDR-grafted antibody. In any aspect or embodiment described herein, FR residues of the human immunoglobulin are replaced by corresponding non-human residues. Furthermore, in any aspect or embodiment described herein, humanized antibodies may comprise residues that are not found in the recipient antibody or in the donor antibody. These modifications may be made to further refine antibody performance. In general, in any aspect or embodiment described herein, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. In any aspect or embodiment described herein, the humanized antibody optionally will also comprise at least a portion of an immunoglobulin constant region (CR) and/or fragment crystallizable region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. The Fc fragment comprises the carboxy-terminal portions of both H chains held together by disulfides. The effector functions of antibodies are determined by sequences in the Fc region; this region is also the part recognized by Fc receptors (FcR) found on certain types of cells.

A “human antibody” is an antibody that possesses an amino acid sequence that corresponds to that of an antibody produced by a human and/or has been made using any of the techniques for making human antibodies as disclosed herein—e.g., an antibody having variable regions in which both the FR and CDR regions are derived from human germline immunoglobulin sequences, and when present, constant region derived from human germline immunoglobulin sequences. The human antibodies of the present disclosure can include amino acid residues not encoded by human germline immunoglobulin sequences (e.g., mutations introduced by random or site-specific mutagenesis in vitro or by somatic mutation in vivo). This definition of a human antibody specifically excludes a humanized antibody comprising non-human antigen-binding residues and/or CDR sequences derived from the germline of another mammalian species have been grafted onto human framework sequences. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “multispecific antibody” is used in the broadest sense and specifically includes, inter alia, an antibody comprising a heavy chain variable domain (V_{H}) and a light chain variable domain (V_{L}), where the V_{H}V_{L} unit has polyepitopic specificity (i.e., the V_{H}V_{L} unit is capable of binding to two different epitopes on one biological molecule or each epitope on a different biological molecule). In any aspect or embodiment described herein, multispecific antibodies include, but are not limited to, full length antibodies, antibodies having two or more V_{L} and V_{H} domains, antibody fragments or derivatives (such as, antigen-binding fragment (Fab), fragment variable (Fv), disulfide-stabilized fragment variable (dsFv), single chain fragment variable (scFv), diabodies, bispecific diabodies and triabodies, linear antibodies (see U.S. Pat. No. 5,641,870, Example 2; Zapata et al., Protein Eng. 8(10): 1057-1062 (1995)), single chain antibody molecules, multispecific antibodies formed from antibody fragments, and/or antibody fragments that have been linked covalently or non-covalently). The expression “linear antibodies” generally refers to the antibodies described in Zapata et al., Protein Eng., 8(10):1057-1062 (1995). Briefly, these antibodies comprise a pair of tandem Fd segments (V_{H}-C_{H1}-V_{H}-C_{H1}) which, together with complementary light chain polypeptides, form a pair of antigen binding regions. Linear antibodies can be bispecific or monospecific.

A “fragment or derivative” of an antibody as used herein in the specification and claims is a protein or glycoprotein which is derived from said antibody and/or comprises a portion of an intact antibody, such as the antigen binding or variable region of an instant antibody. Thus, in any aspect or embodiment described herein, a fragment or derivative of an antibody herein refers to a functional fragment or derivative of an antibody, such as a chimeric antibody and/or multipsecific antibody. In any aspect or embodiment described herein, the fragment or derivative of an antibody comprises a heavy chain variable region. It has been shown that the antigen-binding function of an antibody can be performed by fragments of a full-length antibody or derivatives thereof. Examples of fragments of an antibody include (i) antigen-binding fragment (Fab), monovalent fragments consisting of the variable region and the first constant domain of the heavy and the variable region and the first constant domain of the light chain; (ii) Fab Prime (Fab′) fragments, a monovalent fragment consisting of the variable region and the constant region of each of the heavy and the light chains and a free sulfhydryl group; (iii) bivalent Fab (F(ab′)₂) fragments, bivalent fragments comprising two Fab fragments linked by a disulfide bridge at the hinge region; (iv) Fd fragments consisting of the variable region and the first constant domain C_{H1} of the heavy chain; (v) variable fragments (Fv) consisting of the heavy chain and light chain variable region of a single arm of an antibody; (vi) scFv fragments, Fv fragments consisting of a single polypeptide chain; (vii) dsFv, Fv fragment wherein the VH-VL heterodimer is stabilized by an interchain disulfide bond between structurally conserved framework positions distant from CDRs; (viii) (Fv)₂ fragments consisting of two Fv fragments covalently linked together; (ix) a heavy chain variable domain; (ix) linear antibodies, and (x) multibodies consisting of a heavy chain variable region and a light chain variable region covalently linked together in such a manner that association of the heavy chain and light chain variable regions can only occur intermolecular but not intramolecular. In any aspect or embodiment described herein, derivatives of an antibody include antibodies that bind to or compete with the same antigen as the parent antibody, but which have a different amino acid sequence than the parent antibody from which it is derived. These antibody fragments and derivatives are obtained using conventional techniques known to those with skill in the art, and the fragments are screened for utility in the same manner as are intact antibodies.

Papain digestion of antibodies produces two identical antigen-binding fragments, called “Fab” fragments, and a residual “Fc” fragment, a designation reflecting the ability to crystallize readily. As discussed herein, the Fab fragment consists of an entire L chain along with the variable region domain of the H chain (_{H}), and the first constant domain of one heavy chain (C_{H1}). Pepsin treatment of an antibody yields a single large F(ab′)2 fragment, which as described herein roughly corresponds to two disulfide linked Fab fragments having divalent antigen-binding activity and is still capable of cross-linking antigen. Furthermore, as described herein, Fab′ fragments differ from Fab fragments by having an additional few residues at the carboxy terminus of the C_{H1} domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

An “variable fragment” or “Fv” consists of a dimer of one heavy-chain variable region and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

A “single-chain Fv” (“sFv” or “scFv”) are antibody fragments that comprise the V_{H} and V_{L} antibody domains connected into a single polypeptide chain. In any aspect or embodiment described herein, the sFv polypeptide further comprises a polypeptide linker between the V_{H} domain and V_{L} domain, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995.

The term “diabodies” refers to small antibody fragments prepared by constructing sFv fragments (see preceding paragraph) with short linkers (about 5 to about 10 residues) between the V_{H} and V_{L} domains, such that inter-chain but not intra-chain pairing of the V domains is achieved, resulting in a bivalent fragment (i.e., fragment having two antigen-binding sites). Bispecific diabodies are heterodimers of two “crossover” sFv fragments in which the V_{H} and V_{L} domains of the two antibodies are present on different polypeptide chains. Diabodies are described more fully in, for example, EP 404 097, WO 93/11161, and Hollinger et al., Proc. Natl. Acad. Sci. USA, 90:6444-6448 (1993).

A target amino acid sequence is “derived” from or “corresponds” to a reference amino acid sequence if the target amino acid sequence shares a homology or identity over its entire length with a corresponding part of the reference amino acid sequence of at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In any aspect or embodiment described herein, a target amino acid sequence which is “derived” from or “corresponds” to a reference amino acid sequence is 100% homologous, or in particular 100% identical, over its entire length with a corresponding part of the reference amino acid sequence. The “homology” or “identity” of an amino acid sequence or nucleotide sequence is preferably determined according to the present disclosure over the entire length of the reference sequence or over the entire length of the corresponding part of the reference sequence that corresponds to the sequence that homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% homologous or identical, especially identical, to the respective amino acid sequences of the parent antibody. In any aspect or embodiment described herein, the antibody derived from (i.e., derivative of) a parent antibody comprises the same CDR sequences as the parent antibody, but differs in the remaining sequences of the variable regions.

By “homology” is meant two or more nucleic acid or amino acid sequences is partially or completely identical. In any aspect or embodiment described herein, the homologous nucleic acid or amino acid sequence has 30%, 40%, 50%, 60%, 70%, 80%, 90%, or 95% sequence similarity or identity to a nucleic acid encoding the reference nucleic acid or amino acid sequence.

“Polyepitopic specificity” refers to the ability to specifically bind to two or more different epitopes on the same or different target(s). “Monospecific” refers to the ability to bind only one epitope. In any aspect or embodiment described herein, the multispecific antibody is an IgG1 form that binds to each epitope with an affinity of 5 μM to 0.001 pM, 3 μM to 0.001 pM, 1 μM to 0.001 pM, 0.5 μM to 0.001 pM or 0.1 μM to 0.001 pM. In any aspect or embodiment described herein, the binding affinity (K_{d}) of the antigen binding region or antibody, or antigen binding fragment thereof, to the target protein is less than about 100 μM, about 10 μM, about 1 μM, about 100 nM, about 10 nM, about 1 nM, about 100 pM, about 10 pM, or about 1 pM. In any aspect or embodiment described herein, the binding affinity (Kd) of the antigen binding region or antibody, or antigen binding fragment thereof, to the target protein is about 1 pM to about 50 nM (e.g., about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 1 pM, about 2 pM, about 3 pM, about 4 pM, about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, any values in between, or a range from any combination of the values). In any aspect or embodiment described herein, the binding affinity (Kd) of the antigen binding region or antibody, or antigen binding fragment thereof, to the target protein is about 1 pM to about 15 nM (e.g., about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, any values in between, or a range from any combination of the values).

The term “isolated antibody” as used herein the refers to an antibody that is substantially free of other antibodies having different antigenic specificities (e.g., an isolated bispecific antibody as described herein that specifically binds the protein of interest, such as the antigen, the first antigen, and/or the second antigen). Moreover, an isolated antibody can be substantially free of other cellular material and/or chemicals.

An “affinity matured” antibody is one with one or more alterations in one or more hypervariable regions (HVRs) thereof, which result in an improvement in the affinity of the antibody for antigen, as compared to a parent antibody that does not possess those alteration(s). In any aspect or embodiment described herein, an affinity matured antigen binding region or antibody, or antigen binding fragment thereof, has nanomolar or even picomolar affinities for the target antigen (e.g., about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 20 nM, about 25 nM, about 30 nM, about 35 nM, about 40 nM, about 45 nM, about 50 nM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, about 20 pM, about 25 pM, about 30 pM, about 35 pM, about 40 pM, about 45 pM, about 50 pM, any values in between, or a range from any combination of the values). Affinity matured antibodies may be produced using certain procedures known in the art. For example, Marks et al., Bio/Technology 10:779-783 (1992) describes affinity maturation by VH and VL domain shuffling. Random mutagenesis of HVR and/or framework residues is described by, for example, Barbas et al., Proc Nat. Acad. Sci. USA 91:3809-3813 (1994); Schier et al., Gene 169:147-155 (1995); Yelton et al., J. Immunol. 155:1994-2004 (1995); Jackson et al., J. Immunol. 154(7):3310-9 (1995); and Hawkins et al., J. Mol. Biol. 226:889-896 (1992).

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonist antibody,” as used herein, is an antibody which partially or fully mimics at least one of the functional activities of a polypeptide of interest (e.g., antigen, the first antigen, and/or the second antigen).

“Growth inhibitory” antibodies are those that prevent or reduce proliferation of a cell expressing an antigen to which the antibody binds.

As used herein, “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein. A codon set typically is represented by 3 capital letters in italics, e.g., NNK, NNS, XYZ, DVK, and the like (e.g., NNK codon refers to N=A/TG/C at positions 1 and 2 in the codon and K=G/T at equimolar ratio in position 3 to encode all 20 natural amino acids). A “non-random codon set”, as used herein, thus refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al., J. Mol. Biol. 296:57-86, 1999); Garrard and Henner, Gene 128:103, 1993). Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the present disclosure, have sequences that allow for hybridization to a variable domain nucleic acid template and also can, but do not necessarily, include restriction enzyme sites useful for, for example, cloning purposes.

An antibody of the present disclosure “which binds” or “binds” an antigen of interest (e.g., antigen, the first antigen, and/or the second antigen) is one that binds the antigen with sufficient affinity for the antibody is useful as a diagnostic and/or therapeutic agent in targeting a protein or a cell or tissue expressing the antigen, and does not significantly cross-react with other proteins. In any aspect or embodiment described herein, the extent of binding of the antibody to a “non-target” protein will be less than about 10% of the binding of the antibody to its particular target protein as determined by fluorescence activated cell sorting (FACS) analysis or radioimmunoprecipitation (RIA) or ELISA. With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10 −6 M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In any aspect or embodiment described herein, the term “specific binding” refers to binding where a molecule binds to a particular polypeptide or epitope on a particular polypeptide without substantially binding to any other polypeptide or polypeptide epitope. In any aspect or embodiment described herein, the “specific binding” for a particular polypeptide or an epitope of a particular peptide target has an affinity (K_{d}) of less than about 100 μM, about 10 μM, about 1 μM, about 100 nM, about 10 nM, about 1 nM, about 100 pM, about 10 pM, or about 1 pM. In any aspect or embodiment described herein, the “specific binding” for a particular polypeptide or an epitope of a particular peptide target has an affinity (K_{d}) of about 1 pM to about 50 nM (e.g., about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 1 pM, about 2 pM, about 3 pM, about 4 pM, about 5 pM, about 6 pM, about 7 pM, about 8 pM, about 9 pM, about 10 pM, about 15 pM, about 20 pM, about 25 pM, about 30 pM, about 35 pM, about 40 pM, about 45 pM, about 50 pM, any values in between, or a range from any combination of the values). In any aspect or embodiment described herein, the “specific binding” for a particular polypeptide or an epitope of a particular peptide target has an affinity (Kd) of about 1 pM to about 15 nM (e.g., about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 1 pM, about 2 pM, about 3 pM, about 4 pM, about 5 pM, about 6 pM, about 7 pM, about 8 pM, about 9 pM, about 10 pM, about 11 pM, about 12 pM, about 13 pM, about 14 pM, about 15 pM, any values in between, or a range from any combination of the values).

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative and exemplary embodiments for measuring binding affinity are described in the following.

In any aspect or embodiment described herein, the “Kd” or “Kd value” according to the present disclosure is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ in PBS. When the plates have dried, 150 l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by using surface plasmon resonance assays using a BIACORE®-2000 or a BIACORE®-3000 (BIAcore, Inc., Piscataway, N.J.) at 25° C. with immobilized antigen CMS chips at \hat{˜}10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 g/ml (\hat{˜}0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN-20™ surfactant (PBST) at 25° C. at a flow rate of approximately 25 μl/min. Association rates (k on) and dissociation rates (k off) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k off/k on. See, e.g., Chen et al., J. Mol. Biol. 293:865-881 (1999). If the on-rate exceeds 10⁶ μM −1 s −1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

An “on-rate,” “rate of association,” “association rate,” or “k on” according to the present disclosure can also be determined as described above using a BIACORE®-2000 or a BIACORE®-3000 system (BIAcore, Inc., Piscataway, N.J.).

“Biologically active” and “biological activity” and “biological characteristics” with respect to a polypeptide of the present disclosure means having the ability to bind to a biological molecule, except where specified otherwise.

“Biological molecule” refers to a nucleic acid, a protein, a carbohydrate, a lipid, and combinations thereof. In one embodiment, the biologic molecule exists in nature.

“Isolated,” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

The amino acid sequences described herein are contiguous amino acid sequences unless otherwise specified.

“Structurally unsimilar” biological molecules according to the present disclosure refers to biological molecules that are not in the same class (protein, nucleic acid, lipid, carbohydrates, etc.) or, for example, when referring to proteins, having less than 60% amino acid identity, less than 50% amino acid identity, less than 40% amino acid identity, less than 30% amino acid identity, less than 20% amino acid identity or less than 10% amino acid identity compared to each other.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity, Fc receptor binding antibody-dependent cell-mediated cytotoxicity (ADCC), phagocytosis, down regulation of cell surface receptors (e.g., B cell receptor), and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. (Proc. Natl. Acad. Sci. USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), can be performed.

The term “therapeutically effective amount” refers to an amount of an antibody or antibody fragment (a) to treat a disease or disorder in a subject in need thereof or (b) capable of achieving a therapeutic effect in a subject in need thereof. For example, a therapeutically effective amount of an antibody or antibody fragment can be the amount that is capable of preventing or relieving one or more symptoms associated with a disease or disorder. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). For example, in the case of tumor (e.g., a cancerous tumor), the therapeutically effective amount of the antibody or antibody fragment that may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibody or antibody fragment may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, the presence or size of metastases, the size of the primary tumor, or the size or number of the blood vessels in angiogenic disorders.

The terms “cancer” and “cancerous” refer to or describe the physiological condition in mammals that is typically characterized by unregulated cell growth. Included in this definition are benign and malignant cancers. By “early-stage cancer” is meant a cancer that is not invasive or metastatic or is classified as a Stage 0, I, or II cancer.

The term “precancerous” refers to a condition or a growth that typically precedes or develops into a cancer.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

An “autoimmune disease” herein is a disease or disorder arising from and directed against an individual's own tissues or a co-segregate or manifestation thereof or resulting condition therefrom.

A “subject” is a vertebrate, preferably a mammal, more preferably a human. Mammals include, but are not limited to, farm animals (such as cows), sport animals, pets (such as cats, dogs and horses), primates, mice, and rats.

Throughout this specification and claims, the word “comprise,” or variations such as “comprises” or “comprising,” will be understood to imply the inclusion of a stated integer or group of integers but not the exclusion of any other integer or group of integers.

### Examples

Bispecific antibodies (bsAbs) are a type of antibody with two binding sites, each targeting a different antigen or epitope. Bispecific antibodies (bsAbs) are highly useful in the treatment of diseases, such as hemophilia A, diabetes, Alzheimer's disease, and various ophthalmological diseases, as well as in tumor immunotherapy. There are currently nine approved bispecific antibodies (bsAbs) worldwide, with over 180 in preclinical development and more than 50 investigated in clinical trials. See, e.g., below Table 1, which shows exemplary bispecific antibody therapeutics that are approved or in the clinic.

TABLE 1
Exemplary bispecific antibody therapeutics that are approved or in the clinic
(adapted from Bispecific Antibodies - Current Status and Prospects, released
9 Mar. 2022 by Biopharma PEG at biochempeg.com/article/252.html).

| | Therapeutic | | |
|---|---|---|---|
| Bispecific Antibody | Indication | Phase | Company |
| Blinatumomab | B-cell acute | Approved | AMGEN (Thousand |
| | lymphoblastic | | Oaks, California) |
| | leukemia | | |
| Emicizumab | Hemophilia A | Approved | ROCHE (Basel, |
| | | | Switzerland) |
| Catumaxomab | Malignant ascites in | Approved | FRESENIUS |
| | ovarian and gastric | | BIOTECH GbmH |
| | cancer | | (Munchen Germany) |
| Mosunetuzumab | Non-Hodgkin's | Phase III | ROCHE (Basel, |
| | lymphoma | | Switzerland) |
| REGN1979 | Non-Hodgkin's | Phase III | REGENERON |
| | lymphoma | | PHARMACEUTICALS |
| | | | (Tarrytown, New York) |
| Blofitamab | Non-Hodgkin's | Phase III | GENENTECH (South |
| | lymphoma | | San Francisco, |
| | | | California) |
| Epcoritamab | Non-Hodgkin's | Phase II | MORPHOSYS |
| | lymphoma | | (Planegg, Germany) |
| PF-06863135 | HER2-positive solid | Phase I | PFIZER (New York, |
| | tumors | | New York) |
| XmAb14045 | Acute myeloid | Phase I | XENCOR (Pasadena, |
| | leukemia and non- | | California) |
| | Hodgkin's | | |
| | lymphoma | | |
| MEDI3039 | Solid tumors | Phase I | ASTRAZENECA |
| | | | (Cambridge, United |
| | | | Kingdom) |
| AMG 420 | Relapsed/refractory | Phase I/II | AMGEN (Thousand |
| | multiple lymphoma | | Oaks, California) |
| AFM13 | Hodgkin lymphoma | Phase I/II | Affirmed |
| CEA-TCB | Solid tumors | Phase I/II | ROCHE (Basel, |
| | | | Switzerland) |
| MEDI9197 | Solid tumors | Phase I/II | AstraZeneca |
| RG6025 | HER2-positive breast | Phase I/II | ROCHE (Basel, |
| | cancer | | Switzerland) |

Bispecific antibodies (bsAbs) have shown great promise in treating a variety of diseases due to their ability to target two different epitopes simultaneously. Bispecific antibodies (bsAbs) have many advantages, including enhanced specificity, modulation of immune responses, targeting of complex disease pathways, overcoming resistance, and the potential for combination therapy. However, bispecific antibody (bsAb) development is challenging due to the complexity of their structure and limitations in current antibody discovery methods. Thus, bispecific antibodies (bsAbs) also have limitations, such as complexity of manufacturing, limited clinical experience, risk of immunogenicity, short half-life, and safety concerns (e.g., such as cytokine release syndrome (CRS) or nephrotic and neurological toxicity).

Hybridoma-based methods, which rely on the fusion of two different hybridoma cell lines, are limited by the heterogeneity of the resulting bispecific antibodies (bsAbs) and the difficulty of engineering them for optimal function. Genetic engineering-based methods, such as the use of single-chain variable fragments (scFv) or Fab fragments, have limitations related to stability, expression, and solubility. Bispecific antibodies (bsAbs) can also have a shorter half-life in vivo than monoclonal antibodies, leading to reduced therapeutic efficacy and increased dosing frequency.

There are many ways to make bispecific antibodies (bsAbs), including: (1) genetic engineering, which involves the fusion of two or more monoclonal antibodies or antibody fragments to create a bispecific antibody (bsAbs); (2) hybrid hybridomas, which are created by fusing two different hybridomas that produce monoclonal antibodies with different specificities; (3) chemical conjugation, which involves covalently linking two monoclonal antibodies or antibody fragments with a linker molecule; (4) knob-and-hole technology, which involves the use of protein scaffolds to link two monoclonal antibodies or antibody fragments together; (5) CrossMab technology, which involves the engineering of a single monoclonal antibody to bind to two different antigens; (6) Bispecific T cell Engagers (BiTEs), which are designed to engage T cells and cancer cells by binding to both CD3 on T cells and a tumor-associated antigen; (7) Tandem scFv, which involves the fusion of two single-chain variable fragments (scFv) to create a bispecific antibody (bsAb); and (8) bsAb fragments created by engineering a single antibody fragment, such as a Fab or scFv, with two different binding specificities.

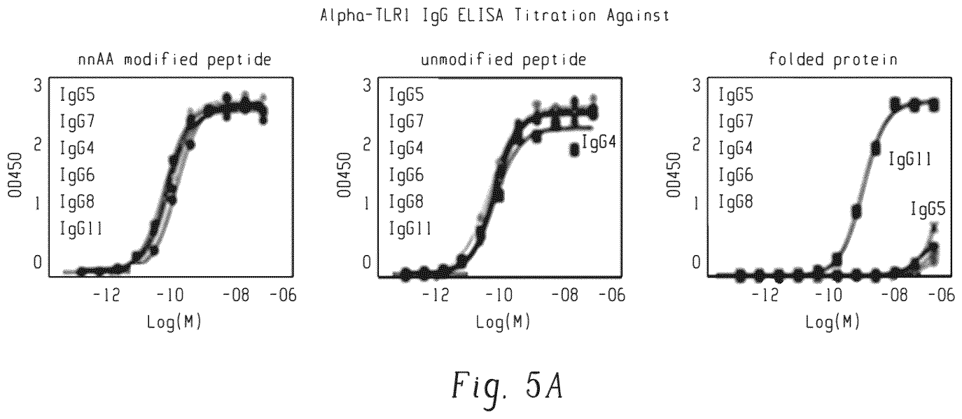

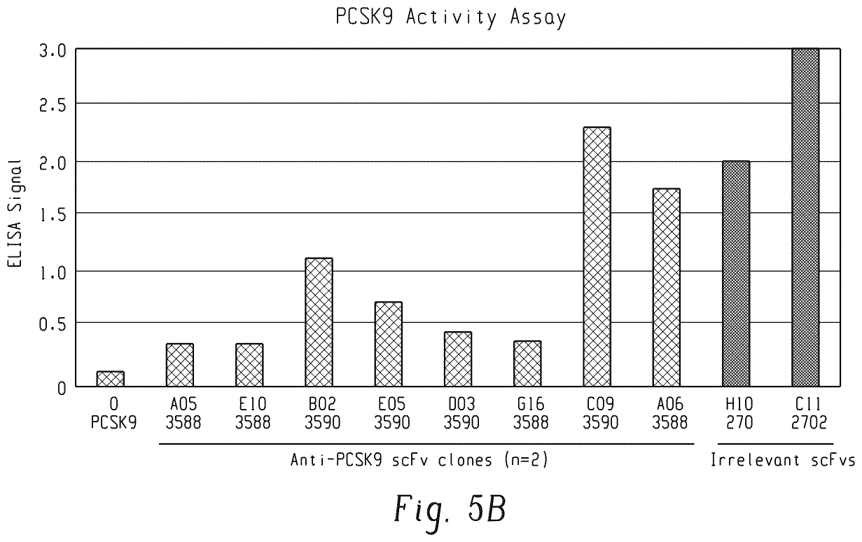

To address some of the limitations of current methods of making bispecific antibodies, a novel approach for generating homodimer IgG bispecific antibodies (bsAbs) using, e.g., site-directed antibody discovery/immunization method (also referred to herein as Epivolve and described in U.S. Patent Application Publication No. 2023/0212271 A1 and Fuller E P, et al. Derivation of splice junction-specific antibodies using a unique hapten targeting strategy and directed evolution. N Biotechnol. 2022 Nov. 25; 71:1-10, which is incorporated by reference in its entirety for all purposes), see FIGS. 5A and 5B, to prepare chimeric antibodies of the present disclosure (also referred to herein as “Shelly”). In any aspect or embodiment described herein, site-specific discovery/immunization method can be used to isolate separate heavy and light chains that bind independently to the same site/epitope on a target protein or two different sites/epitopes on a target protein, which can be engineered into antigen binding regions of an antibody or an antigen binding fragment thereof, such as a bispecific homodimer IgG. This method allows for the selection of antibody pairs with high binding specificity and affinity to two epitopes, which is crucial for the development of effective bispecific antibodies (bsAbs). Furthermore, by directing the immune response to specific sites/epitopes on a target protein, the method described herein allows for the isolation of highly specific and effective therapeutic antibodies, expanding the range of proteins that can be targeted, and offering advantages over current antibody discovery methods.

The novel approach for generating a site-specific homodimer bispecific antibodies (bsAbs) or multispecific antibodies (also referred to herein as Shelly antibodies) described herein, can use the site-specific discovery/immunization method (also referred to herein as Epivolve) and/or a method for examining specificity and/or affinity of an antigen region (also referred to herein as Sundae) is described and demonstrated herein. The specific bispecific antibodies (bsAbs) being made as part of this project will be intra-molecular—i.e., two specific sites on the same protein. Proof-of-concept is demonstrated with the (1) binding and avidity effect of chimeric antibodies directed to the D site in the membrane-proximal external region (MPER) region of Human Immunodeficiency Virus envelope glycoprotein (Env) in Example 1 and (2) blocking the function of the furin protease in proteins involved in furin-dependent diseases.

This approach allows for the selection of heavy and light chain pairs that have high binding specificity and affinity, which is essential for the development of effective bispecific antibodies (bsAbs). These chimeric antibodies can overcome the limitations of current bispecific antibody (bsAb) development and manufacturing methods, as well as contributes to the advancement of the field of antibody engineering and the development of novel therapeutics. The use of the site-directed antibody discovery/immunization method offers several advantages over traditional hybridoma-based or genetic engineering-based methods, including increased binding specificity and affinity, and thus the potential for improved therapeutic efficacy. The development of chimeric antibodies (such as bsAbs) of the present disclosure also provides new opportunities for the treatment of diseases (such as, HIV and furin-dependent diseases), leading to improved patient outcomes and a reduced burden on the healthcare systems.

### Example 1. Examining Chimeric Antibodies Having Non-Natural Combinations of Heavy and Lights Chains that Independently Bind to Different Epitopes

Heavy and light chains that bind specifically and independently to two different sites/epitopes on the target protein are isolated. Next, the heavy and lights chains that bind specifically and independently to two different sites/epitopes are engineered into chimeric homodimer IgG bispecific antibodies (bsAbs).

Therapeutic antibodies that have functional activity, such as inhibition of an enzyme active site or blocking the interaction of two proteins, have several advantages in the development of targeted therapies for various diseases. One major advantage of antibodies having functional activity is their ability to directly interfere with the disease-causing process at the molecular level, leading to a more effective and specific therapy. See, e.g., Table 2 below, which is a list of exemplary therapeutic antibodies with inhibitory functionality. For example, Imatinib is a therapeutic antibody that inhibits the activity of the kinase enzyme BCR-ABL, which is associated with chronic myeloid leukemia. There are several therapeutic antibodies that block protein-protein interactions, leading to the inhibition of disease progression. For example, Adalimumab is a monoclonal antibody that binds to and inhibits the activity of tumor necrosis factor-alpha (TNF-α), a protein involved in inflammatory processes associated with autoimmune diseases.

TABLE 2
Exemplary therapeutic antibodies with inhibitory functionality

| | Therapeutic | | Disease | |
|---|---|---|---|---|
| Antibody | Target | Mode of Action | Indication | Company |
| Adalimumab | Tumor necrosis | Binds and | Rheumatic | ABBVIE |
| | factor-alpha | neutralized TNF-α | arthritis, | (North |
| | (TNF-α) | | psoriasis, | Chicago, |
| | | | Crohn's disease | Illinois) |
| Bevacizumab | Vascular | Binds and inhibits | Colorectal | GENENTECH |
| | endothelial | VEGF | cancer, lung | (South San |
| | growth factor | | cancer, kidney | Francisco, |
| | (VEGF) | | cancer | California) |
| Cetuximab | Epidermal | Binds and block the | Colorectal | Eli |
| | growth factor | EGFR | cancer, head and | Lilly/Merck |
| | receptor (EGFR) | | neck cancer | KGaA |
| Eculizumab | Complement | Binds and inhibits | Paroxysmal | ALEXION |
| | Component 5 | complement | nocturnal | PHARMA |
| | (C5) | component 5 | hemoglobinuria | (Boston, |
| | | | (PNH) | Massachusetts |
| Infliximab | TNF-α | Binds and | Rheumatoid | JANSSEN |
| | | neutralized TNF-α | arthritis, | BIOTECH |
| | | | psoriasis, | (Beerse, |
| | | | Crohn's disease | Belgium) |
| Nivolumab | Programmed | Blocks PD-1 receptor | Melanoma, non- | BRISTOL- |
| | cell death | on T-cells, enhancing | small cell lung | MYERS |
| | protein 1 (PD-1) | anti-tumor activity | cancer, renal | SQUIBB |
| | | | cell carcinoma | (New York, |
| | | | | New York) |
| Omalizumab | Immunoglobulin | Binds and | Asthma, chronic | GENENTECH |
| | E (IgE) | neutralized IgE | idiopathic | (South San |
| | | | urticaria | Francisco, |
| | | | | California)/ |
| | | | | NOVARTIS |
| | | | | (Basel, |
| | | | | Switzerland) |
| Palivizumab | Human | Binds and | Respiratory | MEDIMMUNE |
| | respiratory | neutralized RSV-F | syncytial virus | (Gaithersburg, |
| | syncytial virus | protein | (RSV) | Maryland) |
| | (RSV) F | | | |
| | glycoprotein | | | |
| | (RSV-F) | | | |
| Rituximab | Cluster of | Depletes B-cells via | Non-Hodgkin's' | GENENTECH |
| | differentiation | antibody-dependent | lymphoma, | (South San |
| | 20 (CD20) | cellular cytotoxicity | chronic | Francisco, |
| | | (ADCC) and | lymphocytic | California) |
| | | complement- | leukemia | |
| | | dependent | | |
| | | cytotoxicity (CDC) | | |
| Trastuzumab | Human | Binds and inhibits | HER2-positive | GENENTECH |
| | epidermal | HER2 | breast cancer | (South San |
| | growth factor | | | Francisco, |
| | receptor 2 | | | California) |
| | (HER2 or erbB- | | | |
| | 2)) | | | |

A second advantage of therapeutic antibodies with functional activity is their potential for increased selectivity and reduced off-target effects. By targeting specific molecular sites or interactions, such antibodies can selectively inhibit disease-causing molecules or pathways, while sparing healthy cells and tissues. This can reduce the risk of off-target effects and minimize unwanted side effects associated with current therapies. Antibodies with functional activity can be engineered to have longer half-lives and improved pharmacokinetics, which can lead to increased efficacy and reduced dosing frequency. This is particularly relevant for chronic diseases that require long-term treatment and management.

The methods of making the chimeric antibodies of the present disclosure address some limitations of current bispecific antibodies (bsAb) development methods and contribute to the advancement of the field of antibody engineering and the development of novel therapeutics (e.g., therapeutics for furin-dependent diseases). The development of homodimer IgG bispecific antibodies (bsAbs) using the site-directed antibody discovery/immunization method offers several potential advantages over other bispecific formats and antibody discovery methods. Also described herein are bispecific antibodies (bsAbs) that target furin-dependent diseases, which have been associated with a range of pathologies, including cancer, viral infections, and cardiovascular diseases.

Site-directed antibody (Ab) discovery/immunization method for generating chimeric antibodies of the present disclosure, including multispecific antibodies, bispecific antibodies, and homodimer IgG bispecific antibodies (bsAbs) is an innovative and novel approach to multispecific antibodies (e.g., bispecific antibodies (bsAbs)) development. The site-directed antibody discovery/immunization method enables the isolation of heavy and light chains that bind independently to two different sites on the target protein, which can then be engineered into a chimeric antibody, such as a multispecific antibody, bispecific antibody, or homodimer IgG bsAbs. The approach described herein allows for the selection of antibody pairs that have high binding specificity and affinity, which is essential for the development of effective multispecific antibodies, such as bispecific antibodies (bsAbs). The methods of making chimeric antibodies described herein, and the associated chimeric antibodies, is a novel targeted means of constructing site-specific multispecific or bispecific antibodies (e.g., homodimeric bispecific antibody) to increase avidity. Furthermore, combining the site-directed antibody discover/immunization method (Epivolve) and chimeric antibody (Shelly) methods described herein has the potential to generate many diverse antibody pairs with high binding specificity and affinity, increasing the likelihood of identifying lead candidates for further development.

Furin cleavage is a crucial step in the entry of many viruses into host cells. Furin is expressed in many cell types, including those targeted by viral infections. The cleavage of viral proteins by furin enables the virus to enter the cell and replicate. Many enveloped viruses, including Influenza, Ebola, and Severe Acute Respiratory Syndrome Coronavirus 2 (SARS-CoV-2), use furin cleavage to activate their surface proteins for cell entry. In the save of SARS-CoV-2, furin cleavage of the spike glycoprotein is necessary for the virus to enter human cells, making it a potential target for antiviral therapies. Understanding the role of furin in viral entry can provide insights into the development of effective treatments for viral infections.

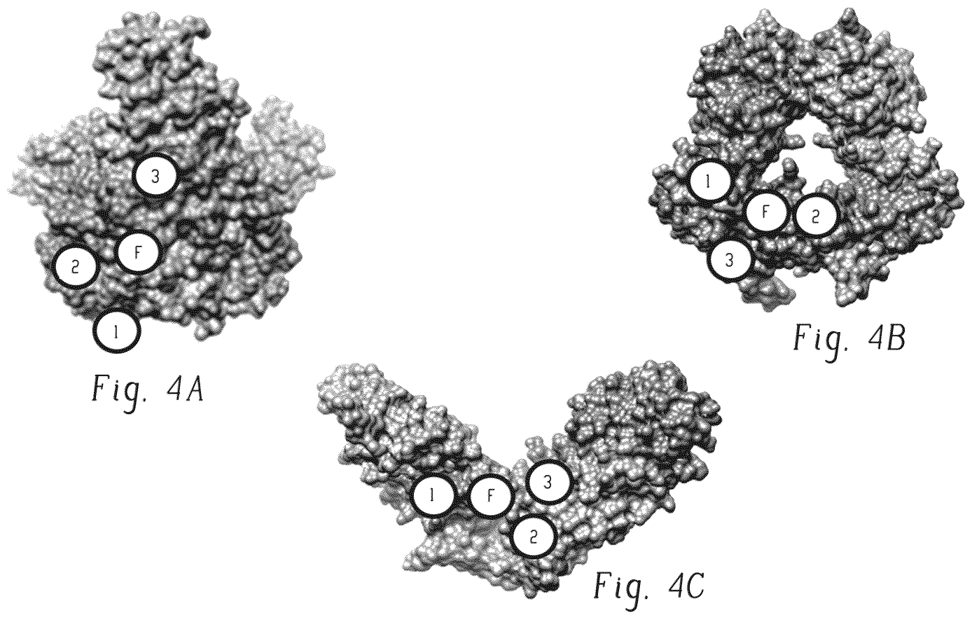

The filovirus surface glycoprotein (GP), shown in FIG. 4A, is a trimeric structure that drives viral entry into the host cell via a complex mechanism that is, to date, only partially understood. The glycoprotein (GP) monomer protein is composed of a glycan cap, and the GP1 and the GP2 subdomains that are cleaved by furin prior to viral entry. Targeted sites 1, 2, and 3 surround the furin cleavage site, “F”, are mapped onto the GP1-GP2 interface. Binding of therapeutic antibodies at sites 1, 2, and 3 are known to block furin cleavage and subsequently, viral entry. Targeted sites 1, 2, and 3 of the TGF-odimer, shown in FIG. 4B, surround the furin cleavage site, “F” (Shi M, Zhu J, Wang R, Chen X, Mi L, Walz T, Springer T A. Latent TGF-β structure and activation. Nature. 2011 Jun. 15; 474(7351):343-9. PDB: 3RJR). Targeted sites 1, 2, and 3 of Pro-myostatin dimer, shown in FIG. 4C, surround the furin cleavage site, “F” (Cotton T R, Fischer G, Wang X, McCoy J C, Czepnik M, Thompson T B, Hyvanen M. Structure of the human myostatin precursor and determinants of growth factor latency. EMBO J. 2018 Feb. 1; 37(3):367-383. PDB: 5NTU).

The present example relates to the demonstration and use of a novel approach for generating homodimer IgG bispecific antibodies (bsAbs) that blocks the function of the furin protease on a targeted protein. Furin is a serine protease that has been implicated in the progression of several diseases, including cancer, viral infections (see Table 3 below), and cardiovascular diseases. Inhibiting the function of the furin protease by sterically blocking the cleavage site may lead to the inhibition of disease progression. The use of a fluorogenic read-out to measure the inhibition of furin cleavage is well-established in the field. This experimental approach allows for the screening of large numbers of bispecific antibody (bsAb) candidates, enabling the rapid identification of suitable clones.

TABLE 3
Furin is Vital for Viral Infectivity

| Virus | Protein | Furin Cleavage Site | Viral Family |
|---|---|---|---|
| Ebola virus | Ebola virus | RRTKR (residues 501-505; | Filoviridae |
| | glycoprotein (GP) | SEQ ID NO: 4) | |
| Marburg virus | Marburg virus | RARR (residues 325-329; | Filoviridae |
| | glycoprotein (GP) | SEQ ID NO: 5) | |
| Lassa fever | Lassa fever virus | RKRK (residues 163-166; | Arenaviridae |
| virus | glycoprotein (GPC) | SEQ ID NO: 6) | |
| Nipah virus | Nipah virus | RKRK (residues 498-501; | Paramyxoviridae |
| | glycoprotein (G) | SEQ ID NO: 6) | |
| Hendra virus | Hendra virus | RKKR (residues 498-501; | Paramyxoviridae |
| | glycoprotein (G) | SEQ ID NO: 7) | |
| Severe Acute | SARS-CoV-2 spike | RRAR (residues 682-685; | Coronaviridae |
| Respiratory | glycoprotein (S) | SEQ ID NO: 8) | |
| Syndrome | | | |
| Coronavirus 2 | | | |
| (SARS-CoV-2) | | | |
| Rift Valley | Rfit Valley fever virus | RRKK (residues 124-127; | Phenuiviridae |
| Fever Virus | glycoprotein (Gn) | SEQ ID NO: 9) | |
| Hantaan Virus | Hataan virus | RRKR (residues 95-98; | Hantaviridae |
| | glycoprotein (Gn) | SEQ ID NO: 10) | |
| Puumala Viru | Puumala virus | RKRR (residues 128-131; | Bimuavorodae |
| | glycoprotein (Gn) | SEQ ID NO: 11) | |
| Crimean-Congo | Crimean-Congo hemorrhagic | RKRR (residues 726-729; | Nairoviridae |
| Hemorrhagic | fever virus glycoprotein (GP) | SEQ ID NO: 11) | |
| Fever Virus | | | |

Chimeric Antibody. Chimeric antibodies of the present disclosure can include homodimer “2 in 1” antibodies, or a second generation “two-in-one” bispecific antibodies, wherein the heavy chain and the light chain each independently interacts with a different epitope, on the same protein or different proteins. These molecules can be based on stochastically, i.e., randomly derived, naturally-occurring antibodies.

However, the present disclosure further contemplates a novel deterministic method, wherein a targeted means of directing the immune response to specific epitopes is utilized. The targeted method of engineer chimeric antibodies of the present disclosure, such as a homodimeric bispecific antibody (bsAb), increases both affinity and avidity of the chimeric antibody.

Site-directed Discovery/Immunization Method. The site-directed discovery/immunization method has been described in U.S. Patent Application Publication No. 2023/02112271 A1 and Fuller E P, et al. Derivation of splice junction-specific antibodies using a unique hapten targeting strategy and directed evolution. N Biotechnol. 2022 Nov. 25; 71:1-10, the contents of which are hereby incorporated by reference in its entirety. The site-directed discovery method (Epivolve) is used for making site-directed antibodies. Briefly, and described elsewhere herein, a highly immunogenic modified ‘mod1’ amino acid (e.g., a peptide comprising a phosphoserine (SEP) at the target site) acts as an immune-target. In vivo method, a first series of immunizations (e.g., a mouse or a rabbit) using a mod1 peptide is followed by a series of immunizations with the native peptide and/or protein. The mod1 peptide immunization first enriches for B cells against the mod1 and the mod1-adjacent sequences on the peptide. The mice are subsequently boosted with a native peptide (or protein, if available). The native-peptide/protein boosting further enriches for B cells producing IgGs that have evolved through somatic hyper-mutagenesis to epitope switch or spread to recognize the native amino acid. Peripheral blood mononuclear cells (PBMCs) are isolated, an IgG is cloned therefrom, and the secreted antibodies validated. In vitro method, a first screen is performed by biopanning a scFv phage library against a mod1 peptide to identify anti-mod1-specific scFv. The anti-mod1-scFv clones can be evolved using a mutagenesis technology, such error prone polymerase chain reaction (EP-PCR or EPP) and/or AXM mutagenesis (Holland E G, Acca F E, Belanger K M, Bylo M E, Kay B K, Weiner M P, Kiss M M. In vivo elimination of parental clones in general and site-directed mutagenesis. J Immunol Methods. 2015 February; 417:67-75). After the mutagenesis, the identified mod1-specific scFvs are then made into a library. The library is then biopanned against the native sequence/peptide or protein. Binding to the native sequence generally requires 1 or 2 amino acid changes to the paratope. Antibodies binding to the native peptide are validated using the full-length protein.

The site-directed discovery/immunization method has several strengths. It is not reliant on having access to the full-length protein, and in vivo, because the initial immune response is always to a highly immunogenic mod1-containing (and therefore non-native) immunogen. The inventors have previously shown that mod1 peptides can overcome tolerance. Antibodies derived from the in vitro and in vivo methods have high binding affinities equivalent to antibodies developed from hybridoma and phage display technologies. Combining the in vivo and in vitro attempts, the site-directed discovery/immunization methods has a success rate of over 85%. Site-directed discovery/immunization method has been used to make anti-splice-site specific antibodies, enzyme inhibitors, G protein-coupled receptor (GPCR) agonists, GPCR antagonists, polymorphism specific antibodies, and pan-variant antibodies (able to recognize 2 or more amino acids at a single site). See FIGS. 5A and 5B.

### Example 2. Optimization and Validation of Site-Directed Antibody Discovery Method for the Isolation of Igg Heavy Chains and Igg Light Chains that Bind Independently to One or More Different Sites on the Target Protein

Develop a workflow amenable to identifying antibodies that recognize a specific site on a targeted protein, testing the resulting heavy chains and light chains for independent binding, making chimeric antibodies from two independently binding complementing heavy and light chains, and test the resulting chimeric antibodies for increased/enhanced binding.

Method: (1) express and purify targeted proteins, if not commercially available; (2) design 3 sets of peptides representing 3 different sites on each of 3 target protein(s) chosen from the list of zymogens in Table 4 according to the site-directed discovery/immunization principles outlined herein—focus on three solvent-exposed sites on the target protein with two sites adjacent to each other and at least one site on the opposite side of a furin cleavage site from the other two sites. Furthermore, preference is given to targets that can be obtained as full-length protein. In vivo or in vitro site-directed discovery/immunization (see FIGS. 5A and 5B) to identify at least five (5) antibodies per site that bind specifically to the intended site on purified target protein. Furthermore, the targets of Table 1 may be utilized. Especially where we can pair up with a lab interested in studying the biology of the target and in need of our reagents. This Aim is low risk and can be accomplished either in vitro or in vivo. An alternative approach is not necessary.

TABLE 4
Zymogens cleaved by furin and commercial availability of zymogens and assays.

| | Precursor to mature | Cleavage of the | |
|---|---|---|---|
| Zymogen | protein | peptide bond between | Substrate Sequence |
| Pro-BDNF | brain-derived neurotrophic | Arg-128 and Met-129 to | Boc-Ala-Ala-Arg- |
| | factor involved in neuronal | generate mature BDNF | AMC |
| | survival and differentiation | | |
| Pro-nerve | nerve growth factor | Arg-218 and Val-219 to | Boc-Asn-Pro-Val- |
| growth factor | involved in neuronal | generate mature NGF | Pro-Asp-Arg-AMC |
| (proNGF) | survival and differentiation | | (SEQ ID NO: 12) |
| Pro-TGF-beta1 | TGFbetal involved in | Arg-278 and Leu-279 to | Abz-Lys-Pro-Leu- |
| | cellular growth, | generate mature TGF- | Ser-Lys-Lys-Val- |
| | differentiation, and | beta1 | Val-Lys(Dnp)-OH |
| | apoptosis | | (SEQ ID NO: 13) |
| Pro-IGF-1 | insulin-like growth factor 1 | Arg-71 and Leu-72 to | Abz-Asn-Leu-Cys- |
| | involved in growth and | generate mature IGF-1 | Tyr-Asp-Ile-Arg-Phe |
| | development | | Arg-Lys(Dnp)-OH |
| | | | (SEQ ID NO: 14) |
| Pro-IGF-2 | insulin-like growth factor 2 | Arg-128 and Ser-129 to | Boc-Asn-Leu-Cys- |
| | involved in growth and | generate mature IGF-2 | Tyr-Asp-Ile-Arg-Phe |
| | development | | Arg-Lys(Dnp)-OH |
| | | | (SEQ ID NO: 15) |
| Pro-endothelin- | endothelin-1 involved in | Trp-21 and Val-22 to | Abz-Trp-Lys-Thr- |
| 1 | vasoconstriction and blood | generate mature | Phe-Tyr-Val-Val- |
| | pressure regulation | endothelin-1 | Gly-Leu-Met-NH₃ |
| | | | (SEQ ID NO: 16) |
| Pro- | Parathyroid hormone- | Arg-20 and Ser-21 to | Boc-Asn-Tyr-Pro- |
| parathyroid | related protein involved in | generate mature PTHrP | Arg-Lys-Arg-Arg- |
| hormone- | calcium homeostasis | | Asn-Lys-Leu-Gly- |
| related protein | | | Lys(Dnp)-NH₂ (SEQ |
| | | | ID NO: 17) |
| Pro-calcitonin | calcitonin involved in | Arg-23 and Ser-24 to | Abz-Ser-Asn-Leu- |
| | calcium homeostasis | generate mature | Asn-Ser-Thr-Asn- |
| | | calcitonin | Tyr-Phe-Phe-Arg- |
| | | | Lys(Dnp)-NH₂ (SEQ |
| | | | ID NO: 18) |
| Pro-atrial | atrial natriuretic peptide | Ser-25 and Gly-26 to | Abz-Ser-Ser-Gln |
| natriuretic | involved in regulation of | generate mature ANP | Asn-Ser-His-Leu- |
| peptide | blood volume and pressure | | Gly-Arg-Ser-Leu- |
| (proANP) | | | Gly-Lys(Dnp)-NH₂ |
| | | | (SEQ ID NO: 19) |
| Pro- | neuropeptide Y involved in | Arg-35 and Tyr-36 to | Abz-Tyr-Pro-Lys- |
| neuropeptide Y | appetite regulation and | generate mature NPY | Pro-Asn-Lys-Tyr- |
| (proNPY) | stress response | | Gly-Leu-Arg-Gln- |
| | | | Arg-Arg-Lys(Dnp)- |
| | | | NH₂ (SEQ ID |
| | | | NO: 20) |
| Coagulation | Cross-linking enzyme | Catalyzes the cross- | The fibrin |
| factor XIII | involved in blood clotting | linking of fibrin, | polymerization |
| | | collagen, and other | inhibitor Gly-Pro- |
| | | proteins | Arg-Pro (SEQ ID |
| | | | NO: 21) |

EXAMPLE 3: Examining a Matrix Composed of Independently Binding Heavy Chains and Independently Binding Light Chains Discovered in Example 3 to Identify Homodimer IgG Bispecific Antibodies (bsAbs). Binding of the bispecific antibodies (bsAbs) will be quantified to triage the IgGs with the appropriate binding characteristics for further validation in a furin cleavage assay in Example 4

Method: (1) clone and co-express a heavy-light chain matrix from the heavy chains and the light chains from the antibodies identified in Example 4 in HEK cells to produce homodimer IgG bispecific antibodies (bsAbs); (2) examiner the chimeric antibodies from the heavy/light chain matrix by ELISA as an initial means to quantify homodimer monospecific- and bispecific IgG antibody binding to peptide and full length (if available) target protein substrate; (3) optionally, characterize the binding kinetics and specificity of the homodimer monospecific- and bispecific IgG antibodies using biophysical and analytical techniques such as surface plasmon resonance (SPR) and Bio-Layer Interferometry (BLI).

### Example 4: Evaluate the Binding Affinity of the Homodimer Igg Bispecific Antibodies (Bsabs) Using an In Vitro Furin Cleavage Assay

Methods: (1) the specificity of the homodimer monospecific- and bispecific IgG antibodies will be assessed and validated in a furin cleavage assay using full-length (FL) protein as a substrate and a means for determining the degree of cleavage, such as gel electrophoreses, column chromatography, or ELISA. Optionally, furin substrates and furin inhibitors can be used in competitive assays to validate antibody blockage. These compounds can also be used as controls.

Therapeutic antibodies represent a promising class of targeted therapies for a wide range of diseases, including cancer, autoimmune disorders, and infectious diseases. Antibodies that inhibit the activity of enzymes or block protein-protein interactions have been shown to block key signaling pathways involved in disease progression, leading to the inhibition of disease growth and spread. The development of novel methods for isolating and characterizing therapeutic antibodies with high binding specificity and affinity, such as phage display technology and site-directed antibody discovery methods, offers exciting opportunities for the advancement of antibody engineering and the development of novel therapeutics. Described herein are methods of developing/engineering multispecific antibodies (such as bispecific antibodies) that target multiple (e.g., 2, 3, 4, 5, 6, 7, 8, 9, or more) different sites on a single protein or different proteins.

### Example 5. Examining Chimeric Antibodies Having Non-Natural Combinations of Heavy and Lights Chains that Independently Bind the D Site in the Membrane-Proximal External Region (MPER) Region of Human Immunodeficiency Virus Envelope Glycoprotein (Env)

The data of Table 5 below demonstrates that chimeric antibodies of the present disclosure (also referred to herein as, Frankenstein antibodies (FnAbs) or Shelly antibodies) are capable of binding the D site in the membrane-proximal external region (MPER) region of Human Immunodeficiency Virus envelope glycoprotein (Env). First, the solubility of the chimeric molecules was examined, and second, the binding of the chimeric molecules to the target protein was evaluated. As shown in Table 5 below, pairing of anti-MPER D heavy chains with independently derived anti-MPER D light chains yielded some chimeric antibodies with an apparent avidity effect. Values in Table 5 are single point enzyme-linked immunosorbent assay (ELISA) OD₄₅₀ values on full length glycoprotein 41 (gp41) using monoclonal Immunoglobulin G (IgG) supernatants generated from Epivolve immunized rabbits for the D664 MPER target. IgG was produced for 21 out of 23 chimeric/Shelly combinations. Combinations with italicized data with a bold box around them are the original non-chimeric/Shelly IgGs. All wells were gp41+ clonotype (bound both the mod1 and NAT D664 peptides as well as the gp41 protein). Clones from different plates (P1, P2, P3, and P4) are from different B-cell wells. Co-expressing a heavy chain and light chain from different plates represents a non-natively paired IgG. Clones P1, P3, and P4 are from one rabbit (CT764) and P2 is from a different rabbit (CT763). Italicized values within the bold boxes are naturally paired heavy and light IgGs. Bold values did not produce soluble IgGs. The HIV MPER sequence with the targeted sites shown below.

MPER:
(SEQ ID NO: 22)
LLELDKWASLWNWFDITNWL

### Other Embodiments

While the inventions of the present disclosure have been described in conjunction with the detailed description thereof, the foregoing description is intended to illustrate and not limit the scope of the invention, which is defined by the scope of the appended claims. Other aspects, advantages, and modifications are within the scope of the following claims. Further, while several embodiments of the present disclosure have been shown and described herein, it will be understood that such embodiments are provided by way of example only. Numerous variations, changes and substitutions will occur to those skilled in the art without departing from the spirit of the invention. Rather, the present disclosure is to cover all modifications, equivalents, and alternatives falling within the scope of the present disclosure as defined by the following appended claims and their legal equivalents. Accordingly, it is intended that the description and appended claims cover all such variations as fall within the spirit and scope of the invention.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients can be varied to optimize the desired effects, additional ingredients can be added, and/or similar ingredients can be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

What is claimed is:

1. An antigen binding polypeptide comprising a heavy chain variable region (VH) coupled to a light chain variable region (VL),
wherein the heavy chain variable region independently binds a first epitope and the light chain variable region independently binds a second epitope.

2. The antigen binding polypeptide of claim 1, wherein;
the first epitope and the second epitope are different epitopes on the same antigen;
the first epitope and the second epitope are on different antigens; or
the first epitope and the second epitope are the same epitope.

3. (canceled)

4. (canceled)

5. A method of making the antigen binding polypeptide of claim 1, the method comprising:
expressing an antigen binding polypeptide comprising a heavy chain variable region (VH) coupled to a light chain variable region (VL),
wherein the heavy chain variable region independently binds a first epitope and the light chain variable region independently binds a second epitope.

6. (canceled)

7. A chimeric antibody or antigen-binding fragment thereof comprising one or more antigen binding region, wherein each of the one or more antigen binding region is independently selected from the antigen binding polypeptide of claim 1.

8. The chimeric antibody or antigen-binding fragment thereof of claim 7, wherein:
the chimeric antibody or antigen-binding fragment thereof comprises two or more independently selected antigen binding polypeptides;
the antibody or antigen-binding fragment thereof is an antibody; or
the antigen-binding fragment of the chimeric antibody is selected from:
(i) an antigen-binding fragment (Fab),
(ii) a Fab prime (Fab′),
(iii) a bivalent Fab (F(ab′)₂),
(iv) a fragment difficult (Fd),
(v) a fragment variable or variable fragment (Fv),
(vi) a single-chain fragment variable (scFv),
(vii) a disulfide-stabilized fragment variable (dsFv),
(viii) a (Fv)₂ fragment,
(ix) a linear antibody, and
(x) a multibody.

9. (canceled)

10. (canceled)

11. A method of making a chimeric antibody or antigen-binding fragment thereof, the method comprising:
expressing a chimeric antibody or antigen-binding fragment thereof comprising one or more antigen binding polypeptides independently selected from claim 1.

12. (canceled)

13. A chimeric antibody comprising two or more antigen binding regions, wherein each antigen binding region is independently selected from the antigen binding polypeptide of claim 1.

14. The chimeric antibody of claim 13, wherein:
each antigen binding region is the same antigen binding region, or
each antigen binding region is a different antigen binding region.

15. (canceled)

16. The chimeric antibody of claim 9, wherein:
each antigen binding region bind the same epitope, or
each antigen binding region bind a different epitope.

17. (canceled)

18. A method of making a chimeric antibody, the method comprising:
expressing a chimeric antibody comprising one or more antigen binding polypeptides independently selected from claim 1.

19. The method of claim 5, further comprising at least one of:
generating one or more antigen binding regions to the first epitope, the second epitope, or both the first epitope and the second epitope;
screening, examining, or determining the binding capacity, affinity, avidity, or a combination thereof, of one or more antigen binding regions for the first epitope, the second epitope, or both the first epitope and the second epitope;
screening, examining, or determining the binding capacity, affinity, avidity, or a combination thereof, of one or more heavy chain variable regions for the first epitope, the second epitope, or both the first epitope and the second epitope;
screening, examining, or determining the binding capacity, affinity, avidity, or a combination thereof, of one or more light chain variable regions for the first epitope, the second epitope, or both the first epitope and the second epitope;
expressing one or more antigen binding polypeptides, antibody comprising two antigen binding polypeptides, antibody fragment comprising one or more antigen binding polypeptides, or combination thereof, wherein each antigen binding polypeptide comprises (i) a heavy chain variable region VH) that independently binds the first epitope coupled to (ii) a light chain variable region (VL) that independently binds the second epitope;
screening, examining, or determining the binding capacity, affinity, avidity, or a combination thereof, of one or more antigen binding polypeptides, antibody comprising two antigen binding polypeptides, antibody fragment comprising one or more antigen binding polypeptides, or combination thereof, wherein each antigen binding polypeptide comprises (i) a heavy chain variable region (VH) that independently binds the first epitope coupled to (ii) a light chain variable region (VL) that independently binds the second epitope; or
a combination thereof.

20. The method of claim 6, wherein at least one of:
generating the one or more antigen binding regions to the first epitope, the second epitope, or both the first epitope and the second epitope, comprises performing site-directed discovery or immunization for the first epitope, the second epitope, or both the first epitope and the second epitope;
screening, examining, or determining the binding capacity, affinity, avidity, or a combination thereof, comprises screening or examining the specificity and/or affinity of the one or more antigen binding regions, the one or more heavy chain variable regions, the one or more light chain variable regions, or a combination thereof, for the first epitope, the second epitope, or both the first epitope and the second epitope; or
a combination thereof.

21. A chimeric antibody comprising:
a first heavy chain variable region (VH1) and a first light chain variable region (VL1), wherein the VH1 and the VL1 each bind a first epitope independently; and
a second heavy chain variable region (VH2) and a second light chain variable region (VL2), wherein the VH2 and the VL2 each bind a second epitope independently.

22. The chimeric antibody of claim 21, wherein:
the first epitope and the second epitope are the same;
the first epitope and the second epitope are different epitopes on the same antigen; or
the first epitope and the second epitope are different epitopes on different antigens.

23. (canceled)

24. (canceled)

25. A chimeric antibody comprising:
a first antigen binding polypeptide comprising a first heavy chain variable region (VH1) and a first light chain variable region (VL1), wherein the VH1 and the VL1 each independently bind an epitope on a first antigen; and
a second antigen binding polypeptide comprising a second heavy chain variable region (VH2) and a second light chain variable region (VL2), wherein the VH2 and the VL2 each independently bind an epitope on an antigen.

26. The chimeric antibody of claim 25, wherein:
the VH1 and the VL1 bind different epitopes on the first antigen, or
the VH1 and the VL1 each independently bind the same epitope on the first antigen.

27. (canceled)

28. The chimeric antibody of claim 25, wherein:
the VH2 and the VL2 bind different epitopes on the antigen, or
the VH2 and the VL2 each independently bind the same epitope on the antigen.

29. (canceled)

30. The chimeric antibody of claim 25, wherein:
the first antigen and the antigen are the same antigen, or
at least one of (i) the VH2 and the VL2 bind epitopes on different antigens, (ii) the first antigen and the antigen are different antigens, or (iii) a combination thereof.

31. (canceled)

32. (canceled)

33. The chimeric antibody of claim 25, wherein the first antigen binding polypeptide is part of a first antigen-binding fragment (Fab), the second antigen binding polypeptide is part of a second antigen-binding fragment (Fab), or a combination thereof.

34. A pharmaceutical composition comprising the antigen binding polypeptide of claim 1.

35. A method of treating a disease or disorder in a subject in need thereof, the method comprising administering the antigen binding polypeptide of claim 1.


SEQUENCE LISTING (22 sequences)

SEQ ID NO: 1 - protein, 4 aa, synthetic construct
GSGS

SEQ ID NO: 2 - protein, 5 aa, synthetic construct
GSGSG

SEQ ID NO: 3 - protein, 6 aa, synthetic construct
GSGSGS

SEQ ID NO: 4 - protein, 5 aa, synthetic construct
RRTKR

SEQ ID NO: 5 - protein, 4 aa, synthetic construct
RARR

SEQ ID NO: 6 - protein, 4 aa, synthetic construct
RKRK

SEQ ID NO: 7 - protein, 4 aa, synthetic construct
RKKR

SEQ ID NO: 8 - protein, 4 aa, synthetic construct
RRAR

SEQ ID NO: 9 - protein, 4 aa, synthetic construct
RRKK

SEQ ID NO: 10 - protein, 4 aa, synthetic construct
RRKR

SEQ ID NO: 11 - protein, 4 aa, synthetic construct
RKRR

SEQ ID NO: 12 - protein, 6 aa, synthetic construct
  MOD_RES 1: note = tert-butyloxycarbonyl
  MOD_RES 6: note = 7-amino-4-methyl coumarin
NPVPDR

SEQ ID NO: 13 - protein, 9 aa, synthetic construct
  MOD_RES 1: note = Aminobenzoic acid
  MOD_RES 9: note = 2,4-Dinitrophenyl
KPLSKYVVK

SEQ ID NO: 14 - protein, 10 aa, synthetic construct
  MOD_RES 1: note = Aminobenzoic acid
  MOD_RES 10: note = 2,4-Dinitrophenyl
NLCYDIRFRK

SEQ ID NO: 15 - protein, 10 aa, synthetic construct
  MOD_RES 1: note = tert-butyloxycarbonyl
  MOD_RES 10: note = 2,4-Dinitrophenyl
NLCYDIRFRK

SEQ ID NO: 16 - protein, 10 aa, synthetic construct
  MOD_RES 1: note = Aminobenzoic acid
  MOD_RES 10: note = Amidated
WKTFYVVGLM

SEQ ID NO: 17 - protein, 12 aa, synthetic construct
  MOD_RES 1: note = tert-butyloxycarbonyl
  MOD_RES 12: note = 2,4-Dinitrophenyl
  MOD_RES 12: note = Amidated
NYPRKRRNKLGK

SEQ ID NO: 18 - protein, 12 aa, synthetic construct
  MOD_RES 1: note = Aminobenzoic acid
  MOD_RES 12: note = 2,4-Dinitrophenyl
  MOD_RES 12: note = Amidated
SNLNSTNYFFRK

SEQ ID NO: 19 - protein, 10 aa, synthetic construct
  MOD_RES 10: note = 2,4-Dinitrophenyl
  MOD_RES 10: note = Amidated
NSHLGRSLGK

SEQ ID NO: 20 - protein, 14 aa, synthetic construct
  MOD_RES 1: note = Aminobenzoic acid
  MOD_RES 14: note = 2,4-Dinitrophenyl
  MOD_RES 14: note = Amidated
YPKPNKYGLRQRRK

SEQ ID NO: 21 - protein, 4 aa, synthetic construct
GPRP

SEQ ID NO: 22 - protein, 20 aa, synthetic construct
LLELDKWASLWNWFDITNWL